(19) United States
(12) Patent Application Publication
(10) Pub. No.: US 2003/0170810 A1
(43) Pub. Date: Sep. 11, 2003

(54) Methods and apparatuses for purification

(76) Inventors: Masoud Vedadi, Toronto (CA); Donald Awrey, Mississauga (CA); Aled Edwards, Toronto (CA); Fabien Marino, Toronto (CA); Matt Narrol, Thornhill (CA); Hai Luong, Toronto (CA); Raymond Hui, Toronto (CA)

Correspondence Address: FOLEY HOAG, LLP, PATENT GROUP, WORLD TRADE CENTER WEST, 155 SEAPORT BLVD, BOSTON, MA, 02110, US

(21) Appl. No.: 10/246,812

(22) Filed: Sep. 18, 2002

Related U.S. Application Data
(60) Provisional application No. 60/323,040, filed on Sep. 18, 2001.
(60) Provisional application No. 60/369,817, filed on Apr. 4, 2002.
(60) Provisional application No. 60/399,984, filed on Jul. 31, 2002.

(51) Int. Cl. C12P 21/06; C12M 1/33; C12M 3/08

(52) U.S. Cl. 435/69.1; 435/306.1

(57) ABSTRACT

Proteins can be purified and concentrated from crude cell lysates using high-throughput, automated process control systems that use software application programs to instruct one or more processors, controllers, and actuators to control and/or monitor the operations of filtration, purification, and concentration modules in such systems. The high-throughput production of concentrated proteins can be enabled by filtering the lysates and using compressed, non-reactive gas to concentrate the purified proteins, rather than using more complex centrifugation techniques.

### RELATED APPLICATION INFORMATION

This application claims the benefit of priority to Provisional Patent Application Nos. 60/323,040, filed Sep. 18, 2001, 60/369,817, filed Apr. 4, 2002, and 60/399,984, filed Jul. 31, 2002, which applications are hereby incorporated by reference in their entirety.

### TECHNICAL FIELD

The disclosed technology relates generally to the purification of cell components and more particularly to the automation of high-throughput processes and systems that purify cell components.

### BACKGROUND

The normal or pathologic function of a particular cell depends, at least in part, on the genes that are expressed by that cell. Cells may differ both in terms of the particular genes expressed and in the relative level of expression of the same gene. Differential gene expression can be manifested, for example, by differences in the expression of proteins encoded by the gene, or in post-translational modifications of expressed proteins. For example, proteins can include carbohydrates or phosphate groups, or they can be processed through peptide cleavage.

The identification and characterization of proteins that are differentially expressed by genes of particular cell types is referred to as proteomics. The comparison of expressed proteins can prove useful in identifying biomolecules that may be responsible for a particular pathologic activity of a cell. For example, identifying a protein that is expressed in cancer cells but not in normal cells is useful for diagnosis and, ultimately, for drug discovery and treatment of the pathology.

Proteomic inquiries into the complex and highly diverse structure and function of proteins require the analysis of purified proteins. The diversity in protein properties, the large number of protein types, and the ability to produce purified samples of such proteins in sufficient amount for experimentation complicates and delays the analysis and burdens the limited resources available to perform such analyses. Accordingly, significant effort is being expended to develop automated processes and systems that enable the parallel processing of potentially diverse proteins and that increase the yield of such purified proteins, so that the rate of protein experimentation and drug discovery can increase.

### SUMMARY OF THE INVENTION

In one aspect, a method for purifying a polypeptide is provided, comprising: obtaining a crude cell lysate; mixing the crude cell lysate with ion exchange resin; applying the mixture to a column and passing the lysate through the column, thereby producing a clarified cell lysate; applying the clarified cell lysate to an affinity column under conditions to promote binding of polypeptides to the column resin; washing the affinity column to remove non-specifically bound polypeptides; and eluting the bound polypeptides from the affinity resin, thereby producing a purified polypeptide.

The disclosed technology enables a process control system to automatically and independently process one or more organic samples in parallel (i.e., beginning processing of each organic sample at substantially the same time), staggered parallel (i.e., parallel processing with the processing of at least some of the organic samples beginning at different times), or serial modes (i.e., processing an organic sample to completion and then beginning the processing of another organic sample) to consistently and reliably yield concentrated samples of targeted biomolecules at desired purity levels. For example, the disclosed methods and systems can be applied to form a concentrated protein solution with a purity exceeding 95% from a crude cell lysate.

In one embodiment, the disclosed systems and methods use a filter to filter out at least a portion of the biomass from a crude cell lysate to form a filtered lysate (or clarified lysate), which is pumped/transferred to at least one purifier (via a tube or other mechanism for transferring fluids) that purifies the filtered lysate into a substantially purified cell component, such as a purified protein solution. In one embodiment, the filtered lysate is formed by mixing a crude lysate with ion exchange resin, applying it a column and allowing the lysate to pass through the column. In another embodiment, the filtered lysate is formed by passing the crude lysate through an ion exchange membrane. The purifier can include an ion exchange module that binds at least a portion of the charged molecules out of the filtered lysate to further fractionate the lysate, an affinity module that binds one or more target proteins (or other cell components) from the lysate and elutes the bound protein (or other bound cell component) using a buffer, and a desalting module that alters the buffer of the eluted protein (or other eluted cell component) to form the purified protein solution (or other purified cell component). The ion exchange module can use at least one of a resin and a membrane when further fractionating the lysate. The purifier can also contain one or more additional purification modules for further fractionation of the sample, such as, gel filtration resins or membranes. One or more mixing modules can also be used to combine a composition with either or both of the filtered lysate and/or purified protein solution. The purified solution is then pumped or otherwise transferred to a concentrator where pressure (e.g., from a compressed gas, which is non-reactive with the purified protein) forces excess liquid (e.g., a buffer) from the purified solution, resulting in a predetermined concentration or volume of the purified protein. In an exemplary embodiment, UV sensors (or other detection device) may be used to feed back information related to the quantity of purified protein to the control processor which can then control the concentrator to make automatic adjustments such that the desired volume or concentration of protein will be obtained.

The disclosed systems and methods can use a software application program with a control process or any other software process operating within a memory of a digital data processing device to provide instructions for execution by one or more processors so as to affect the operation of the filter, purifier, and/or concentrator. The processor generates signals in response to the executed instructions, which cause one or more controllers to communicate with actuators (e.g., pumps, flow and pressure valves, etc.) to selectively affect the operation of the filter, purifier, and/or concentrator. For example, the processor can instruct a controller corresponding to a particular flow valve to configure the flow valve in a particular configuration that affects the operation of the purifier and/or concentrator and/or the corresponding controller can specify a particular pump operation (e.g., on, off, flow rate, flow direction).

The controllers can also receive operational parameters associated with the filter, purifier, and/or concentrator from sensors capable of sensing such parameters. These sensors can generate signals corresponding to these operational parameters that are, for example, indicative of a level associated with the filtered lysate, an amount of excess liquid in the concentrator, and/or a concentration of the concentrated protein in the concentrator. The controllers can provide these operational parameters to the processor, which can, for example, instruct a pump to transfer or stop the filtered lysate, start or stop the pressurization/depressurization of the concentrator in response to the amount of excess liquid, and/or resolve error conditions associated with the operation of the filter, purifier, and/or concentrator (e.g., an obstruction in a tube associated with the fluid coupling of the filter, purifier and/or concentrator), without requiring an input from a user of the processor. The processor can also affect the operation of the filter, purifier, and/or concentrator to perform a self-cleaning process, without requiring an input from the user of the processor. The processor can further affect the temperature, humidity and/or other environmental conditions of the filter, purifier, concentrator, and/or of any solutions contained therein by instructing environmental subsystems to maintain desired temperature (e.g., within 0.5 degrees Celsius within a range between about 2 and 4 degrees Celsius) and humidity levels (e.g., below 60%).

The software application program can also use a messaging process and/or a display process, capable of communicating with the control process, to provide indicia of the operation of the filter, purifier, and/or concentrator to a remote user and/or a graphical user interface, respectively, in response to a signal from the control process.

In one embodiment, the disclosed systems and methods purify protein samples using an array of filter columns, an array of purifier columns, and an array of concentrators, all in fluid communication with each other. Individual filter columns, purifier columns and concentrators from the arrays of such elements can be arranged to form one or more channels that enable independent and parallel preparation of purified proteins. Accordingly, a plurality of channels can be formed from the arrays of filter, purifier, and concentrator elements, where each channel prepares a purified protein at substantially the same time and without interaction between the various channels. The array of purifier columns can include an ion exchange column (e.g., an anion exchange column such as DE52), an affinity column (e.g., an IMAC column such as Ni—NTA), and/or a desalting column (e.g., a gel filtration column such as Sephadex G25) and these same column types can be included within one or more channels. The columns may comprise chromatography resins or chromatography membranes. The ion exchange column binds at least a portion of the charged molecules out of a lysate filtered by one of the filter columns to form a further fractionated lysate (or ion exchange flow through). The affinity column binds a protein from the ion exchange flow through and elutes the bound protein using a buffer. The desalting column alters the buffer of the eluted protein to form the purified protein. In certain embodiments, the purifier may additionally comprise one or more additional chromatography columns, such as a gel filtration column, to further fractionate the protein sample. One or more mixing modules may be included before and/or after one or more of the columns for addition of reagents that are desired to be added to the protein solution. The disclosed methods and systems of this embodiment can also include a plurality of actuators, at least one processor, at least one controller, and/or a plurality of sensors interacting as previously described.

### BRIEF DESCRIPTION OF THE DRAWINGS

The foregoing discussion will be understood more readily from the following detailed description of the invention, when taken in conjunction with the accompanying drawings in which:

FIG. 1 schematically illustrates an automated process control system suitable for the high-throughput purification and concentration of one or more organic samples;

FIG. 2 illustrates an exemplary methodology used in operating the automated process control system of FIG. 1;

FIG. 3 schematically illustrates an exemplary embodiment of the automated process control system of FIG. 1 pertaining to the purification and concentration of a protein; and

FIG. 4 illustrates an exemplary methodology used in operating the automated process control system of FIG. 3.

### DETAILED DESCRIPTION

The invention provides methods, particularly automated methods, for purifying cell components, e.g., nucleic acids, proteins, peptides, lipids, polysaccharides, and combinations thereof, from cells. The cell components can be endogenous to the cell, i.e., naturally present in the cell, or they can be exogenous or heterologous to the cell, i.e., not naturally present in the cell. In one embodiment, the invention provides methods for purifying recombinant proteins produced in a cell.

The methods can be used for purifying components from a cell of any origin, e.g., prokaryotic or eukaryotic origin, such as bacterial cells, mammalian cells, yeast cells, insect cells, plant cells, viruses, etc. The methods can be used to purify any type of cell component, e.g., a protein, a nucleic acid (e.g., mRNA, rRNA, tRNA, mitochondrial RNA, and DNA), a lipid, a sugar or combination or derivative thereof.

In a particular embodiment, the invention provides an automated procedure for clarifying a cell lysate and purifying a component from the lysate.

1. Definitions

For convenience, certain terms employed in the specification, examples, and appended claims are collected here. Unless defined otherwise, all technical and scientific terms used herein have the same meaning as commonly understood by one of ordinary skill in the art to which this invention belongs. These terms should be understood and read in light of the specification as a whole.

The articles “a” and “an” are used herein to refer to one or to more than one (i.e., to at least one) of the grammatical object of the article. By way of example, “an element” means one element or more than one element.

The term “automated” refers to a method or process that requires minimal user intervention.

The term “cell” is intended to encompass prokaryotic cells, eukaryotic cells, phage particles, and organelles.

The terms “cell culture” or “culture” include any combination of cells and medium. The cells need not be actively growing.

The term “crystallization buffer” refers to a buffer that is suitable for storage or further manipulation of a substantially purified protein. For example, crystallization buffer may comprise salt and a buffering agent, and any other components that may help maintain protein stability under a variety of conditions.

The terms “lysate” or “cell lysate” refer to a composition comprising at least some cells that have ruptured cell walls and/or cell membranes. “Crude lysate” refers to a lysate that has not been fractionated to remove one or more cellular components. A crude lysate refers to a lysate wherein less than 50%, 20%, 15%, 10%, 5%, or 1% of the biomass has been removed from the mixture. “Clarified lysate” refers to a cell lysate that has been fractionated to remove one or more cellular components, such as cell debris and other insoluble materials, cell wall and/or cell membrane materials, lipids, insoluble proteins, nucleic acids, including DNA and RNA. A clarified cell lysate refers to a lysate wherein at least 1%, 5%, 10%, 15%, 20%, or 50%, or more, of the biomass in the lysate has been removed by fractionation of the lysate. Fractionation of a lysate may be accomplished, for example, by centrifugation, filtration, chromatography, etc. A clarified lysate may also be referred to herein as a filtered lysate. A “fractionated lysate” refers to a clarified lysate that has been further fractionated to remove at least a portion of one or more components in the clarified lysate. In an exemplary embodiment, a fractionated lysate refers to a clarified lysate that has been passed over an ion exchange column to remove at least a portion of the charged molecules in the lysate.

“Lysing,” with reference to a cell suspension, refers to rupturing the cell walls and/or cell membranes of at least a portion of the cells such that at least part of the contents of the cells are released. Lysis may be conducted by the use of a variety of agents, e.g., alkali, detergents, organic solvents and enzymes; by mechanical action, e.g., by sonication and French press; and/or by subjecting the cell to a particular condition, e.g., heat and/or pressure.

“Non-human animals” include mammals such as rodents, non-human primates, ovines, bovines, canines, felines, chickens, amphibians, reptiles, etc.

“Nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

“Cell culture density” refers to the concentration of cells in a solution (e.g., cells/mL).

“External manipulation,” with reference to cell culture density, refers to any alteration of the cell concentration other than by growth of the cells. Examples of external manipulation include, for example, concentration of cells by centrifugation and reduction of culture volume or filtration to remove some or all of the culture medium.

“Harvesting” refers to an alteration of the environmental conditions of a cell culture in order to decrease the rate of growth or protein production of the culture. The rate of growth or protein production may be altered, for example, by changing the temperature, aeration rate, or both, of the culture.

“Not significantly increased,” with reference to a cell culture density upon harvesting, indicates that the concentration of cells in the culture is not increased by more than about 1.1, 1.2, 1.5, or 2 fold after harvesting the cells.

“Protein” (when single chain), “polypeptide” and “peptide” are used interchangeably herein. In certain instances, a protein may comprise two or more polypeptide chains that are associated through covalent or non-covalent interactions.

“Recombinant protein” refers to a protein that is produced by recombinant DNA techniques. For example, a nucleic acid encoding a polypeptide may be inserted into a suitable expression vector which is in turn used to transform a host cell suitable for expression of the polypeptide.

“Substantially pure” refers to an object species that is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 to 90 percent of all macromolecular species present in the composition. Most preferably, the object species is purified to essential homogeneity (contaminant species cannot be detected in the composition by conventional detection methods) wherein the composition consists essentially of a single macromolecular species.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No.: 4,683,195; Nucleic Acid Hybridization (B. D. Hames & S. J. Higgins eds. 1984); Transcription And Translation (B. D. Hames & S. J. Higgins eds. 1984); Culture of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986).

2. Synthesis of Recombinant Proteins

In one embodiment, the invention provides a method for isolating proteins from cells. In a particular embodiment, the protein is recombinantly produced in the cell. Recombinant proteins can be expressed in cells according to methods known in the art, e.g., as described below, which generally involve introducing a nucleic acid encoding the protein of interest into a host cell and culturing the host cell under conditions suitable for expression of the protein of interest.

Nucleic acids encoding polypeptides of interest can be obtained from commercial sources or it can be prepared according to methods known in the art. For example, a nucleic acid encoding a polypeptide of interest can be obtained by reverse transcriptase polymerase chain reaction (RT-PCR) using primers based on the nucleotide sequence of the gene encoding the polypeptide of interest. Alternatively, nucleic acids can be isolated by hybridization. Nucleic acids encoding fusion polypeptides, e.g., a polypeptide of interest fused to an affinity tag, can be prepared by methods known in the art (see, for example, Current Protocols in Molecular Biology, eds. Ausubel et al. John Wiley & Sons: 1992).

In one embodiment, the nucleic acid encoding a polypeptide of interest is operably linked to at least one transcriptional control sequence, e.g., a promoter and an enhancer. Generally, such nucleic acids are also incorporated into a plasmid or an expression vector, which is then introduced into a host cell to allow expression of the fusion polypeptide. The type of transcriptional control sequences used will depend on the particular expression system used, e.g., whether the system is prokaryotic (e.g., bacterial) or eukaryotic (e.g., yeast, avian, insect or mammalian). Mammalian transcriptional control elements are described, e.g,. in Transcriptional regulatory sequences are described in Goeddel; Gene Expression Technology: Methods in Enzymology 185, Academic Press, San Diego, Calif. (1990). Expression systems and appropriate transcriptional control sequences are further described below.

A nucleic acid encoding a polypeptide of interest can modified such as to increase its rate of expression in a particular cell. For example, certain codons can be changed to codons that are highly used in the particular expression system used for expressing the polypeptide (“codon bias”). Such substitutions are known to improve the expression of polypeptides in particular systems.

Suitable vectors for the expression of polypeptides in prokaryotic cells, such as E. coli, include plasmids of the types: pBR322-derived plasmids, pEMBL-derived plasmids, pEX-derived plasmids, pBTac-derived plasmids and pUC-derived plasmids.

In one embodiment, the powerful phage T5 promoter, which is recognized by E. coli RNA polymerase, is used together with a lac operator repression module to provide tightly regulated, high level expression or recombinant proteins in E. coli. In this sytem, protein expression is blocked in the presence of high levels of lac repressor. Such vectors are available commercially, e.g., from Qiagen (Chatsworth, Calif.) (QIAexpress pQE vectors). The nucleic acid encoding the polypeptide of interest can also be operably linked to an inducible or constitutive bacterial promoter, e.g., the anaerobic E. coli, NirB promoter or the E. coli lipoprotein llp promoter, described, e.g., in Inouye et al. (1985) Nucl. Acids Res. 13:3101; Salmonella pagC promoter (Miller et al., supra), Shigella ent promoter (Schmitt and Payne, J. Bacteriol. 173:816 (1991)), the tet promoter on Tn10 (Miller et al., supra), the ctx promoter of Vibrio cholera, the iron-regulated promoters of FepA and TonB (Headley, V. et al. (1997) Infection & Immunity 65:818; Ochsner, U. A. et al. (1995) Journal of Bacteriology 177:7194; Hunt, M. D. et al. (1994) Journal of Bacteriology 176:3944; Svinarich, D. M. and S. Palchaudhuri. (1992) Journal of Diarrhoeal Diseases Research 10:139; Prince, R. W. et al. (1991) Molecular Microbiology 5:2823; Goldberg, M. B. et al. (1990) Journal of Bacteriology 172:6863; de Lorenzo, V. et al. (1987) Journal of Bacteriology 169:2624; and Hantke, K. (1981) Molecular & General Genetics 182:288).

The nucleic acid encoding the polypeptide of interest and the bacterial promoter to which it is operably linked are preferably in a vector or plasmid. As used herein, the term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/or expression of nucleic acids contained therein. Vectors capable of directing the expression of genes to which they are operatively linked are referred to herein as “expression vectors.” The term “plasmid” as used herein, refers generally to circular double stranded DNA loops which are not bound to the chromosome. In the present specification, “plasmid” and “vector” are used interchangeably as the plasmid is the most commonly used form of vector.

Other prokaryotic host cells, in addition to E. coli, include, for example, Shigella spp., Salmonella spp., Listeria spp., Rickettsia spp., Yersinia spp., Escherichia spp., Klebsiella spp., Bordetella spp., Neisseria spp., Aeromonas spp., Franciesella spp., Corynebacterium spp., Citrobacter spp., Chlamydia spp., Hemophilus spp., Brucella spp., Mycobacterium spp., Legionella spp., Rhodococcus spp., Pseudomonas spp., Helicobacter spp., Vibrio spp., Bacillus spp., and Erysipelothrix spp. Most of these bacteria can be obtained from the American Type Culture Collection (ATCC; 10801 University Blvd., Manassas, Va. 20110-2209).

Preferred mammalian expression vectors contain both prokaryotic sequences, to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papillomavirus (BPV-1), or Epstein-Barr virus (pHEBo, pREP-derived and p205) can be used for transient expression of proteins in eukaryotic cells. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2^{nd} Ed., ed. by Sambrook, Fritsch and Maniatis (Cold Spring Harbor Laboratory Press: 1989) Chapters 16 and 17.

Suitable mammalian host cells include but are not limited to VERO, HeLa, mouse C127, Chinese hamster ovary (CHO), WI-38, BHK, COS, and MDCK cell lines. Particularly preferred host cells are CHO cell lines deficient in dihydrofolate reductase such as ATCC CRL 1793, CRL 9096.

Generally, a nucleic acid encoding a polypeptide of interest is introduced into a host cell, such as by transfection, and the host cell is cultured under conditions allowing expression of the fusion polypeptide. Methods of introducing nucleic acids into prokaryotic and eukaryotic cells are well known in the art. Suitable media for mammalian and prokaryotic host cell culture are well known in the art. Generally, the nucleic acid encoding the polypeptide of interest is under the control of an inducible promoter, which is induced once the host cells containing the nucleic acid have divided a certain number of times. For example, where a nucleic acid is under the control of a beta-galactose operator and repressor, isopropyl beta-D-thiogalactopyranoside (IPTG) is added to the culture when the bacterial host cells have attained a density of about OD₆₀₀ 0.45-0.60. The culture is then grown for some more time to give the host cell the time to synthesize the polypeptide. Cultures are then typically frozen and may be stored frozen for some time, prior to isolation and purification of the polypeptide.

A number of vectors exist for the expression of recombinant proteins in yeast. For instance, YEP24, YIP5, YEP51, YEP52, pYES2, and YRP17 are cloning and expression vehicles useful in the introduction of genetic constructs into S. cerevisiae (see, for example, Broach et al. (1983) in Experimental Manipulation of Gene Expression, ed. M. Inouye Academic Press, p. 83, incorporated by reference herein). These-vectors can replicate in E. coli due to the presence of the pBR322 ori, and in S. cerevisiae due to the replication determinant of the yeast 2 micron plasmid. In addition, drug resistance markers such as ampicillin can be used.

In some instances, it may be desirable to express the polypeptide of interest in a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

In cases where plant expression vectors are used, the expression of a polypeptide of interest may be driven by any of a number of promoters. For example, viral promoters such as the 35S RNA and 19S RNA promoters of CaMV (Brisson et al., 1984, Nature, 310:511-514), or the coat protein promoter of TMV (Takamatsu et al., 1987, EMBO J., 6:307-311) may be used; alternatively, plant promoters such as the small subunit of RUBISCO (Coruzzi et al., 1994, EMBO J., 3:1671-1680; Broglie et al., 1984, Science, 224:838-843); or heat shock promoters, eg., soybean hsp 17.5-E or hsp 17.3-B (Gurley et al., 1986, Mol. Cell. Biol., 6:559-565) may be used. These constructs can be introduced into plant cells using Ti plasmids, Ri plasmids, plant virus vectors; direct DNA transformation; microinjection, electroporation, etc. For reviews of such techniques see, for example, Weissbach & Weissbach, 1988, Methods for Plant Molecular Biology, Academic Press, New York, Section VIII, pp. 421-463; and Grierson & Corey, 1988, Plant Molecular Biology, 2d Ed., Blackie, London, Ch. 7-9.

An alternative expression system which can be used to express a polypeptide of interest is an insect system. In one such system, Autographa californica nuclear polyhedrosis virus (AcNPV) is used as a vector to express foreign genes. The virus grows in Spodoptera frugiperda cells. The nucleotide sequence encoding the polypeptide of interest may be cloned into nonessential regions (for example the polyhedrin gene) of the virus and placed under control of an AcNPV promoter (for example the polyhedrin promoter). Successful insertion of the coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed. (e.g., see Smith et al., 1983, J. Virol., 46:584, Smith, U.S. Pat. No. 4,215,051).

In certain embodiments, proteins that may be purified according to the methods and apparatuses of the invention may be fusion proteins containing a domain which increases the solubility and/or facilitates the purification, identification, detection, and/or structural characterization of the protein. Exemplary domains, include, for example, glutathione S-transferase (GST), protein A, protein G, calmodulin-binding peptide, thioredoxin, maltose binding protein, HA, myc, poly arginine, poly His, poly His-Asp or FLAG fusion proteins and tags. Additional exemplary domains include domains that alter protein localization in vivo, such as signal peptides, type III secretion system-targeting peptides, transcytosis domains, nuclear localization signals, etc. In various embodiments, polypeptides may comprise one or more heterologous sequences or domains. Polypeptides may contain multiple copies of the same heterologous sequences or may contain two or more different heterologous sequences. The fusion with the heterologous sequence may occur at the N-terminus of the polypeptide, at the C-terminus of the polypeptide, or at both the N- and C-terminus of the polypeptide. Linker sequences may also be included between a polypeptide and the heterologous sequence in order to facilitate construction of the fusion protein or to optimize protein expression or structural constraints of the fusion protein. In another embodiment, the polypeptide may be constructed so as to contain protease cleavage sites between the fusion polypeptide and polypeptide of the invention in order to remove the tag after protein expression and purification. Examples of suitable endoproteases, include, for example, Factor Xa and TEV proteases.

In another embodiment, proteins that may be purified according to the methods and apparatuses of the invention may be labeled with an isotopic label to facilitate its detection and or structural characterization using nuclear magnetic resonance or another applicable technique. Exemplary isotopic labels include radioisotopic labels such as, for example, potassium-40 (⁴⁰K), carbon-14 (¹⁴C), tritium (³H), sulphur-35 (³⁵S), phosphorus-32 (³²p), technetium-99m (^{99m}Tc), thallium-201 (²⁰¹Tl), gallium-67 (⁶⁷Ga), indium-111 (¹¹¹In), iodine-123 (¹²³I), iodine-131 (¹³¹I), yttrium-90 (⁹⁰Y), samarium-153 (¹⁵³Sm), rhenium-186 (¹⁸⁶Re), rhenium-188 (¹⁸⁸Re), dysprosium-165 (¹⁶⁵Dy) and holmium-166 (¹⁶⁶Ho). The isotopic label may also be an atom with non zero nuclear spin, including, for example, hydrogen-1 (¹H), hydrogen-2 (²H), hydrogen-3 (³H), phosphorous-31 (³¹P), sodium-23 (²³Na), nitrogen-14 (¹⁴N), nitrogen-15 (¹⁵N), carbon-13 (¹³C) and fluorine-19 (¹⁹F). In certain embodiments, the polypeptide is uniformly labeled with an isotopic label, for example, wherein at least 50%, 70%, 80%, 90%, 95%, or 98% of the possible labels in the polypeptide are labeled, e.g., wherein at least 50%, 70%, 80%, 90%, 95%, or 98% of the nitrogen atoms in the polypeptide are ¹⁵N, and/or wherein at least 50%, 70%, 80%, 90%, 95%, or 98% of the carbon atoms in the polypeptide are ¹³C, and/or wherein at least 50%, 70%, 80%, 90%, 95%, or 98% of the hydrogen atoms in the polypeptide are ²H. In other embodiments, the isotopic label is located in one or more specific locations within the polypeptide, for example, the label may be specifically incorporated into one or more of the leucine residues of the polypeptide. The invention also encompasses the embodiment wherein a single polypeptide comprises two, three or more different isotopic labels, for example, the polypeptide comprises both ¹⁵N and ¹³C labeling.

In yet another embodiment, proteins that may be purified according to the methods and apparatuses of the invention may be labeled to facilitate structural characterization using x-ray crystallography or another applicable technique. Exemplary labels include heavy atom labels such as, for example, cobalt, selenium, krypton, bromine, strontium, molybdenum, ruthenium, rhodium, palladium, silver, cadmium, tin, iodine, xenon, barium, lanthanum, cerium, praseodymium, neodymium, samarium, europium, gadolinium, terbium, dysprosium, holmium, erbium, thulium, ytterbium, lutetium, tantalum, tungsten, rhenium, osmium, iridium, platinum, gold, mercury, thallium, lead, thorium and uranium. In an exemplary embodiment, polypeptides are labeled with seleno-methionine.

A variety of methods are available for preparing a polypeptide with a label, such as a radioisotopic label or heavy atom label. For example, in one such method, an expression vector comprising a nucleic acid encoding a polypeptide is introduced into a host cell, and the host cell is cultured in a cell culture medium in the presence of a source of the label, thereby generating a labeled polypeptide. As indicated above, the extent to which a polypeptide may be labeled may vary.

Isotopic substitution may be accomplished by growing a host cells, transformed by genetic engineering to produce the protein of choice, in a growth medium containing ¹³C-, ¹⁵N- and/or ²H-labeled substrates. In certain instances, bacterial growth media consists of ¹³C-labeled glucose and/or ¹⁵N-labeled ammonium salts dissolved in D₂O where necessary. Kay, L. et al., Science, 249:411 (1990) and references therein and Bax, A., J. Am. Chem. Soc., 115, 4369 (1993). More recently, isotopically labeled media especially adapted for the labeling of bacterially produced macromolecules have been described. See U.S. Pat. No. 5,324,658. The goal of these methods has been to achieve universal and/or random isotopic enrichment of all of the amino acids of the protein. By contrast, methods allow only certain residues to be relatively enriched in ¹H, ²H, ¹³C and ¹⁵N. For example, Kay et al., J. Mol. Biol., 263, 627-636 (1996) and Kay et al., J. Am. Chem. Soc., 119, 7599-7600 (1997) have described methods whereby isoleucine, alanine, valine and leucine residues in a protein may be labeled with ²H, ¹³C and ¹⁵N, but specifically labeled with ¹H at the terminal methyl position. Similarly, a cell-free system has been described by Yokoyama et al., J. Biomol. NMR, 6(2), 129-134 (1995), wherein a transcription-translation system derived from E. coli was used to express human Ha-Ras protein incorporating ¹⁵N serine and/or aspartic acid. Techniques for producing isotopically labeled proteins and macromolecules, such as glycoproteins, in mammalian or insect cells have been described. See U.S. Pat. Nos. 5,393,669 and 5,627,044; Weller, C. T., Biochem., 35, 8815-23 (1996) and Lustbader, J. W., J.Biomol. NMR, 7, 295-304 (1996). Other methods for producing polypeptides and other molecules with labels appropriate for NMR are known in the art.

For production of heavy atom labeled proteins, it is possible to replace endogenous light metals in metallo-proteins with heavier ones, e.g., zinc by mercury, or calcium by samarium (Petsko, G. A., 1985. Methods in Enzymology, Vol. 114. Academic Press, Orlando, pp. 147-156). Exemplary sources for such heavy compounds include, without limitation, sodium bromide, sodium selenate, trimethyl lead acetate, mercuric chloride, methyl mercury acetate, platinum tetracyanide, platinum tetrachloride, nickel chloride, and europium chloride.

3. Cell Lysis

In one embodiment, cell lysates may be prepared by pelleting the cells (e.g., by centrifugation or filtration) and removing part or all of the medium in which they had been grown prior to rupturing the cell wall and/or cell membrane. Lysis of pelleted cells can be conducted according to methods well known in the art, such as by the use of a variety of agents, e.g., alkali, detergents (e.g. CHAPS and SDS), organic solvents and enzymes (e.g. lysozyme); by mechanical action, e.g., by sonication and French press; and/or by subjecting the cell to a particular condition, e.g., heat and/or pressure and/or a freeze/thaw cycle. Other agents can be added after lysis, e.g., Triton X-100 for facilitating solubilization of proteins. For example, for isolating proteins from prokaryotic cells, the cells can be lysed in the presence of CHAPS, e.g., at about 0.1 g/ml and optionally in the presence of protease inhibitors, e.g., at about 100 μg/ml and/or Benzonase, e.g., about 500 units (Novagen, 25 units/μl). The cells may then be incubated with a rocking motion, e.g., for about 45 minutes at room temperature. In a further step, the lysate can be sonicated, e.g., once for 30 seconds at output 4 to 5 at a duty cycle of about 80%.

In another embodiment, cell lysates useful for purification of cellular components, including nucleic acids and proteins, may be prepared by adding detergent to a cell culture and sonicating the culture. In an exemplary embodiment, the density of the cell culture upon harvesting is not significantly increased by external manipulation prior to sonication of the culture. For example, the density of the cell culture is not significantly increased by concentrating the cells through centrifugation or filtration and removal of part or all of the medium in which the cells had been grown. In certain embodiments, cell lysis may include a freeze thaw cycle either before and/or after addition of the detergent. Additionally, lysis may include an incubation step (for example at 4° C., 25° C. or 37° C. for 5-60 minutes) with or without agitation, such as rocking. Lysis can be carried out in the cell culture medium itself. Alternatively, the medium can be changed prior to lysis. Sonication may be carried out as described above for cell pellets. Suitable detergents include, for example, CHAPS, NP-40, SDS, or Triton X-100, at a final concentration of about 0.01% to about 2.0%, or about 0.1% to about 1.0% (w/v) detergent. In an exemplary embodiment, CHAPS is added to a final concentration 0.5% (w/v) prior to sonication. In certain embodiments, cells to be lysed are present in a medium at a concentration of at least about 10⁴, 10⁵, 10⁶, 10⁷, 10⁸, 10⁹ or 10¹⁰ cells/ml. The concentration of cells in the culture (or culture density) refers to the number of cells in a given volume when well mixed and is not meant to account for local variations throughout the culture. For example, a culture having 10⁶ cells/mL when well mixed will not change its density merely by letting the cell culture sit for a certain time so that the cells settle to the bottom of the container (e.g., thus having a higher local density at the bottom of the container). A culture density may be increased by removal of at least a portion of the liquid volume of the culture while maintaining substantially the same total number of cells. For example, the liquid volume may be reduced by evaporation or by pelleting the cells and decanting at least a portion of the supernatant.

4. Lysate Clarification

In one embodiment, crude cell lysates may be clarified by fractionation of the lysate to remove at least a portion of the biomass in the lysate. For example, lysates may be clarified by centrifugation to remove any unlysed cells and at least a portion of the insoluble cellular debris. The supernatant forms the clarified lysate and may be removed and subjected to further purification processes.

In another embodiment, a crude cell lysate may be clarified by mixing the lysate with an anion exchange resin in batch and then separating the insoluble material (including the resin and anything bound thereto, cells and cell debris) from the soluble material via filtration, centrifugation or gravity separation. In an exemplary embodiment, the crude cell lysate/resin mixture is applied to an empty column (e.g., a column with a frit but without resin) and the soluble material is allowed to pass through the column producing a clarified lysate. The column may optionally be washed with an appropriate buffer. The clarified cell lysate may then be subjected to further purification. In one embodiment, the ion exchange resin is an anion exchange resin, such as, for example, DE52. The resin may be mixed with the crude cell lysate at a ratio of about 0.1 g to 10 g, or about 0.5 g to 2 g, or about 1 g, of resin (dry weight) for each 10 mL of cell culture used to produce the crude cell lysate. In certain embodiments, the crude cell lysate may be diluted with buffer at a ratio of about 1:1 to 1:5, about 1:1 to 1:3, about 1:1 to 1:2 (volume/volume) before or after adding the resin.

In another embodiment, clarified lysate may be produced by passing a crude cell lysate over an ion exchange membrane (such as Mustang Q from Pall Biopharmaceuticals, Inc.). The membrane may optionally be washed with an appropriate buffer.

5. Protein Purification

In one embodiment for purifying a protein or polypeptide, a cell lysate is subjected to chromatography. The cell lysate may or may not be first clarified to remove part of the biomass in the lysate. In various embodiments, chromatography may be carried out using chromatography resins and/or chromatography membranes.

In one embodiment, a lysate is subjected to ion exchange chromatography, e.g., using resins. Ion exchange chromatography involves the interaction of charged functional groups in the sample with ionic functional groups of opposite charge on an adsorbent surface. Exemplary resins include sepharose resins, Sephadex resins, Streamline resins, and Source resins from Amersham-Pharmacia Biotech; HyperD resins, Trisacryl resins, and Spherosil resins from BioSepra; TSKgel resins and Toyopearl resins from TosoHaas; Fractogel EMD resins from Merck; Poros resins from Perseptive Biosystems; Macro-Prep resins from BioRAD; and Express-ion resins from Whatman. The ion exchange chromatography may use anionic or cationic substituents attached to matrices, e.g., resins, in order to form anionic or cationic supports for chromatography. “Cation exchange resins” refers to ion exchange resins with covalently bound negatively charged ligands that are free to interact with couterions in a solution with which the resin is contacted. A wide variety of cation exchange resins are known in the art, e.g., those wherein the covalently bound groups are carboxylate or sulfonate. Cationic exchange substituents include carboxymethyl (CM), sulfoethyl (SE), sulfopropyl (SP), phosphate (P) and sulfonate (S). Commercially available cation exchange resins include CMC-cellulose, SP-Sephadex™, and Fast S-Sepharose™ (the latter two being commercially available from Pharmacia). “Anion exchange resins” refers to ion exchange resins with covalently bound positively charged groups, such as quaternary amino groups. Anionic exchange substituents include diethylaminoethyl (DEAE), quaternary aminoethyl (QAE) and quaternary amine (Q) groups. Commercially available anion exchange resins include DEAE cellulose, QAE Sephadex™, and Fast Q Sepharose™ (the latter two being commercially available from Pharmacia). Cellulosic ion exchange resins such as DE23, DE32, DE52, CM-23, CM-32 and CM-52 are available from Whatman Ltd. Maidstone, Kent, U. K. SEPHADEX™-based and cross-linked ion exchangers are also known. For example, DEAE-, QAE-, CM-, and SP-SEPHADEX™ and DEAE-, Q-, CM-and S-SEPHAROSE and SEPHAROSE™ Fast Flow are all available from Pharmacia AB. Further, both DEAE and CM derivitized ethylene glycol-methacrylate copolymer such as TOYOPEARL DEAE-650S or M and TOYOPEARL CM-650S or M are available from Toso Haas Co., Philadelphia, Pa.

Ion exchange chromatography can also be carried out using inorganic adsorbents. Inorganic substances that can be used for protein adsorption, include oxides, insoluble hydroxides, and phosphates. Examples of inorganic materials that have been used for protein purification include Alumina gel Cγ (gel and crystalline), Bentonite (a silicaceous powder), Titanium oxide (TiO₂), Zinc hydroxide gel, and calcium phosphate in the form of Aged gel, Brushite and Hydroxyapatite. Inorganic materials are particularly useful for industrial applications, since they generally are cheaper than other types of chromatographic material. A polypeptide binds to inorganic materials at least through electrostatic interaction. The interaction could also be a polar dipole-dipole bonding.

Hydroxyapatite can be obtained commercially from several companies, e.g., Bio-Rad. Alternatively, it can be prepared, e.g., by slowly mixing together 0.5M solutions of CaCl₂ and Na₂HPO₄ with a solution of 1M NaCl, which results in the production of brushite, CaHPO₄.2H₂O. Boiling of the brushite with NaOH, converts it to hydroxyapatite, Ca₁₀(PO₄)₆(OH)₂ (Bernardi et al. (1971) Methods Enzymol. 22:325).

Ion exchange chromatography can be carried out using a batch or column-type system. In one embodiment, a lysate or polypeptide solution (or preparation) is incubated together with an ion exchange resin in a batch-type system. After a short incubation, preferably on ice, the molecules that are not binding to the resin are recovered by separating out the resin from the solution. This can be done, e.g., by centrifugation to pellet the resin, by gravity sedimentation, or by filtration. Alternatively, a polypeptide solution may be poured over a column containing an ion exchange resin or membrane. The bound or unbound fractions may then be recovered.

The choice of starting pH, buffer and ionic strength for use in ion exchange chromatography is done according to well-known techniques such as conventional test-tube methods, as described, e.g., handbooks from Amersham-Pharmacia Biotech and in Robert K. Scopes, Protein Purification, Principles and Practice, Third Ed., Springer Verlag N.Y., 1993. The chromatographic ion exchange resin is chosen depending on the specific polypeptide of interest to be purified and the conditions employed, such as pH, buffer, ionic strength etc., which are known to the person skilled in the art. Typically, a pH below the isoelectric point (pI) of the polypeptide of interest is used for cation exchange resins and a pH above the pI of the polypeptide of interest is used for anion exchange resins. In addition, a buffer having a sufficient buffer strength to maintain the desired pH is used.

High capacity resins are preferred, and preferably resins of larger particle size, e.g., in the range 100 to 300 μm. The increased bed stability of large beads is of advantage in treating viscous materials, such as cell lysates allowing minimal back pressure when the process is carried out by column chromatography.

In one embodiment, a protein is purified by affinity chromatography, e.g., following an ion exchange chromatography. Affinity chromatography relies on the interaction of the protein with an immobilized ligand. In certain embodiments, a polypeptide may be linked to an “affinity tag,” i.e., a molecule, e.g., a peptide, that binds specifically to another molecule, e.g., with a constant of dissociation (Kd) of at least about 10⁻⁶; 10⁻⁷; 10⁻⁸; 10⁻⁹; 10⁻¹⁰; 10⁻¹¹; 10⁻¹²M. An affinity tag may be linked to the N- or C-terminus of a polypeptide of interest, inserted within the polypeptide of interest or linked to one or more amino acids within the polypeptide of interest.

Affinity tags include those that can be used in immobilized metal affinity chromatography (IMAC). For example, an affinity tag can be a polyhistidine sequence, for example, a hexahistidine sequence (6×His), which interacts specifically with metal ions such as Fe, Co, Ni, Cu, Zn, and Al ions. The affinity tag may also comprise at least 1, 2, 3, 4, 5, 6, 7, 8, 9, 10, 11, 12 or more histidine residues. The affinity tag can also be a polylysine or polyarginine sequence, comprising at least about 4, 5, 6, 7 or more lysine or arginine residues, respectively, which interact specifically with zinc, copper or, for example a zinc finger protein. A His₆ polypeptide tag is uncharged at physiological pH. In addition, it rarely alters or contributes to protein immunogenicity, and rarely interferes with protein structure or function, and does not interfere with protein secretion (Sisk et al. (1994) J. Virol. 68:766). The sequences and affinity purification conditions are well known in the art. Vectors for producing fusion proteins contain such sequences and matrices to which they bind are commercially available. For example, the following kits provide vectors and matrices for purifying proteins containing His tags: QIAexpress Ni—NTA Protein Purification System of Qiagen (Qiagen, Calif.); HAT™ Protein Expression & Purification System (Clontech, Palo Alta, Calif.); pTrcHis Xpress™ Kit (InVitrogen); and BugBuster™ His•Bind® Purification Kit (Novagen). In one embodiment, the resin is Superflow Ni—NTA from Qiagen (#30450) or Talon Cellthru Cobalt resin from Clonetech (#8910-2).

IMAC relies on the formation of weak coordinate bonds between metal ions immobilized on a column and basic groups on proteins, mainly histidine residues. The adsorbent can be formed by attaching to the matrix a suitable spacer arm plus a simple metal chelator, usually based on imino diaetate structures, e.g., in the form of EDTA (see Robert K. Scopes, supra). Imino diacetate (IDS) and tris(carboxymethyl) ethylene diamine (TED) can be used. These chelating ligands will bind tightly to metal ions, in particular to the divalent ions of the transition metals Fe, Co, Ni, Cu, and Zn, but also trivalent metal ions (Fe and Al).

Although histidine residues on proteins are most attracted to immobilized metals, other potential electron donating side chains include tryptophan and cysteine (at neutral pH). Binding to an affinity column requires that these amino acids are located on the surface of a polypeptide and the strength of interaction depends on the number of such linkages for the size of the polypeptide.

Metal chelate adsorbents are generally provided without the metal. Accordingly, in a first step, the column is loaded with the metal. In one embodiment, a solution containing, e.g., 50 mM of the metal salt, e.g., CuSO₄, Zn acetate or NiCl₂, is passed through the column until it is saturated with the metal. Excess metal ions are then washed out. This is preferably followed by a wash with a weak complexing agent, such as 1-10 mM imidazole or 0.5M glycine. In an even more preferred embodiment, the column is further washed with a more concentrated solution of the weak complexing agent, e.g., from about 10 to about 100 mM imidazole, preferably from about 20 to about 80 mM imidazole, and even more preferably about 60 mM imidazole. It may also be desirable to wash the column with the solution that will be used for eluting the polypeptide of interest from the column to avoid having metal ions leach out into the final preparation.

IMAC is generally performed at high ionic strength, e.g., in 100 mM-1M NaCl, e.g., to avoid ion exchange effects. Application buffers generally are at pH 6 to 8, not containing complexing agents. The polypeptide solution is slowly passed through the column, generally at 4° C., e.g., at a flow rate of about 1 ml/minute. The column is then washed with start buffer until all unbound proteins are eluted. Elution of the polypeptide of interest can be achieved by either of two methods. According to one method, the polypeptide is eluted by a stronger complexing agent, e.g., imidazole or EDTA. In the other method, the polypeptide is eluted by lowering the pH of the buffer, such that, e.g., the histidines on the polypeptide become protonated, and so are unable to coordinate with the metal ions. In certain embodiments, a combination of methods is used for eluting the polypeptide of the invention from the metal affinity matrix.

Accordingly, in one embodiment, an eluate or flow through from an ion exchange chromatography column containing a polypeptide of interest fused to an affinity tag for use in IMAC, is loaded onto a chelating sepharose column charged with NiCl₂. After loading, the column may be washed to remove unbound proteins. Another wash including 60 mM imidazole can be made to wash out unspecifically bound proteins. The histidine tagged polypeptide can then be eluted by the addition of about 200 to 400 mM imidazole, preferably about 250-350 mM imidazole. The preferred amount of imidazole may depend on the particular polypeptide that is purified.

In certain embodiments, the affinity tag may be cleaved from the polypeptide using an endonuclease after purification on an affinity column. The cleaved affinity tags may then be removed from the protein solution by gel filtration chromatography or by passing the solution over a second affinity column so that the cleaved tags will bind to column and the polypeptide will pass through.

The imidazole can then be removed from the solution containing the polypeptide of interest by dialysis or by passing it over a desalting column. The preparation of the recombinant protein can further be purified by conducting a second IMAC. This second IMAC can be run as the first one. Yet further rounds of purification can be undertaken.

The amount of polypeptide of interest in a sample can be determined by a Coomassie protein assay according to the manufacturer's instructions (Bio-Rad). The amount and purity of proteins can also be determined by subjecting the protein mixtures to SDS-PAGE, optionally followed by Western blot analysis. SDS-PAGE gels can be stained, e.g., silver or Coomassie blue stained, for visualizing polypeptides. Western blots can also be incubated with a reagent binding specifically to the polypeptide of interest, e.g., an antibody. The inclusion of known amounts of reference proteins permit, by comparison, to estimate the quantity of a particular protein on the Western blot.

In another embodiment, the affinity tag is maltose binding protein (MBP) or a portion thereof sufficient to bind to another molecule. In an even more preferred embodiment, the other molecule is amylose or an analog thereof, e.g., an analog that can bind to maltose binding protein. Maltose binding protein binds to amylose, and the interaction can be disrupted with maltose or a maltose analog. The nucleic acid sequence encoding maltose binding protein can be found, e.g., as GenBank Accession No. AE000476. Maltose binding protein affinity reagents are available from New England Biolabs (see, e.g., www.neb.com/).

In another embodiment, the affinity tag is glutathione S transferase (GST) polypeptide, which can be prepared, e.g., by using pGEX prokaryotic expression vectors from Pharmacia (Piscataway, N.J.). When using GST fusion proteins, resin linked to GST (Sigma Chem. Co.; St. Louis, Mo.), to glutathione or to an antibody specific for GST can be used, e.g., GST sepharose 4B column (Pharmacia-LKB) or mouse anti-GST-Sepharose® 4B, available from, e.g., Zymed Laboratories. Protein purification can be done as described, e.g., in Kuge et al. (1997) Protein Science 6: 1783 and in Tian et al. (1993) Cell 74:105. Systems for expressing and purifying recombinant proteins comprising a GST tag are available from Novagen as BugBuster™ GST•Bind™ Purification Kit and GST•Tag™ Assay Kit.

Yet other affinity tags include a Self-Cleavable Chitin-binding Tag, e.g., as available from New England Biolabs as the IMPACT™-TWIN System and IMPACT™-CN System; a T7 tag are available from Novagen as T7•Tagg® Purification Kit; an S tag or thioredoxin (trxA), which are available from Novagen. Yet another affinity tag is a cellulose-binding protein A from Clostridium cellulovorans (see, eg., Shpigel et al. (2000) Biotechnol. Appl. Biochem. 31:197).

In certain embodiments, it may be desirable to include a protease or enterokinase cleavage site located between the tag and the polypeptide of interest, allowing the removal of the heterolgous polypeptide after affinity purification (see, e.g., see Hochuli et al. (1987) J. Chromatography 411:177; and Janknecht et al. PNAS 88:8972). An exemplary protease recognition site is a Factor Xa protease recognition site. A vector including a sequence encoding a stretch of six histidines and a site susceptible to a protease is available, e.g., from Qiagen (Chatsworth, Calif.) (pQE-30 Xa Vector containing a Factor Xa Protease recognition site). Another protease cleavage site is a thrombin cleavage site that can be cleaved by thrombin or a TEV protease cleavage site that is cleaved by the TEV protease.

In other embodiments, the affinity tag is an antigen and an antibody to the antigen is used for affinity purifying the affinty tagged polypeptide. In other embodiments, the polypeptide of interest is fused to a tag that consists of, or comprises, an antibody (e.g., a single chain antibody) and the affinity purification comprises using an antigen to which the antibody binds specifically. In yet other methods, avidin and biotin are used.

In certain embodiments, the ion exchange and/or affinity chromatography is preceded or followed by (immediately after or after certain other purification steps) by another form of chromatography. In an illustrative embodiment, further ionic exchange chromatography, size exclusion chromatography or hydrophobic interaction chromatography (HIC) is used after or before the ion exchange chromatography. Size exclusion chromatography, otherwise known as gel filtration or gel permeation chromatography, relies on the penetration of macromolecules in a mobile phase into the pores of stationary phase particles. Size exclusion chromatographic supports based on cross-linked dextrans, e.g., SEPHADEX™, spherical agarose beads e.g. SEPHAROSE™ (both commercially available from Pharmacia AB, Uppsala, Sweden); cross-linked polyacrylamides e.g. BIO-GEL™ (commercially available from BioRad Laboratories, Richmond, Calif.); or ethylene glycol-methacrylate copolymer e.g. TOYOPEARL HW65S (commercially available from ToyoSoda Co., Tokyo, Japan) can be used.

HIC is based on a hydrophobic interaction between the solute and the matrix. Hydrophobic interactions are strongest at high ionic strength, therefore, this form of separation is conveniently performed following salt precipitations or ion exchange procedures. Elution from HIC supports can be effected by alterations in solvent, pH, ionic strength, or by the addition of chaotropic agents or organic modifiers, such as ethylene glycol. A description of the general principles of hydrophobic interaction chromatography can be found in U.S. Pat. No. 3,917,527 and in U.S. Pat. No. 4,000,098. The application of HIC to the purification of specific proteins is exemplified by reference to the following disclosures: human growth hormone (U.S. Pat. No. 4,332,717), toxin conjugates (U.S. Pat. No. 4,771,128), antihemolytic factor (U.S. Pat. No. 4,743,680), tumor necrosis factor (U.S. Pat. No. 4,894,439), interleukin-2 (U.S. Pat. No. 4,908,434), human lymphotoxin (U.S. Pat. No. 4,920,196) and lysozyme species (Fausnaugh, J. L. and F. E. Regnier, J. Chromatog. 359:131-146 (1986)).

In a preferred embodiment, the protein sample is subjected to a method for desalting. This can be accomplished, e.g., by passing the protein sample over Sephadex G25 fine resin (e.g., Amersham Pharmacia Biotech AB (#17-0032-02 exclusion range 1 to 5 Kda). In addition to remove salt from the protein preparation, this step is useful for removing glycerol, imidazole and other impurities, e.g., nickel ion and EDTA. In other embodiments, this step can be replaced by dialysis.

The protein sample may further be concentrated according to methods known in the art, e.g., using concentrators, e.g., UltraFree-0.5 Centrifugal Filter Device-Millipore (5,000 MWCO or 10,000 MWCO) (UFV2BGC40). In another embodiment, centrifugation may be used to concentrate protein. In a exemplary embodiment, pressure (e.g., using pressurized non-reactive gas, such as nitrogen or argon gas) is used to concentrate the proteins by pushing a portion of buffer from the protein solution through a filter that does not allow the protein to pass through.

In other embodiments, the methods and apparatuses described herein may be used for the purification of a variety of cell components in addition to polypeptides. For example, nucleic acids, lipids, carbohydrates, and various other small organic molecules may be purified in accordance herewith. The skilled artisan will be able to determine the appropriate purification scheme for such molecules based on the teachings herein.

In exemplary embodiments, the methods and apparatuses described herein may use chromatography membranes in place of chromatography resins for protein purification. Various membranes suitable for a variety of purification processes are commercially available, such as ion exchange and affinity membranes. Such membranes may be disposable or re-usable and may be single or multi-layered. The membranes typically are housed in self-contained plastic or stainless steel units. An example of an ion exchange membrane is the Mustang Q ion exchange membrane available from Pall Biopharmaceuticals, Inc. (East Hills, N.Y.). See also http://domino.pall.com and www.labfilters.com. The skilled artisan will readily be able to adapt the methods and apparatuses disclosed herein for use with chromatography membranes based on the teachings herein. In one embodiment, a method for clarifying a cell lysate involves applying a crude cell lysate (that optionally may first be diluted with buffer at a ratio of about 1:1 or 1:3) to an ion exchange membrane and passing the lysate through the membrane, thereby producing a clarified cell lysate. The membrane may optionally be washed with buffer.

In various embodiments, protein purification may be conduct using one or more chromatography columns. Lysates or protein solutions may be passed over individual columns or the columns may be arranged so that the flow through or eluate from one column loads directly onto another column (e.g., the columns are arranged in tandem). When using multiple columns in tandem, the columns may be fluidly connected and solutions may be passed through the system by means of a pump or vacuum. In an exemplary embodiment, proteins are purified using a combination of an ion exchange column (e.g., an anion exchange column, such as DE52), an affinity column (e.g., an IMAC column, such as Ni—NTA), and a gel filtration column (e.g., a desalt column, such as Sephadex G25). Optionally, the purification scheme may also comprise additional chromatography steps, such as an additional gel filtration step to further fractionate the sample and/or an additional affinity chromatography step (e.g., to remove cleaved affinity tags). At various stages during the protein purification process, various chemicals, enzymes or small molecules may be added to the purification sample to aid in purification or downstream processing, or to modify the sample. For example, reagents that may be added to the sample include, for example, EDTA, proteases (e.g., thrombin), protein co-factors or ligands, etc.

In a preferred embodiment of the invention, a method for purifying a protein of interest comprises one or more of the following steps: (i) providing a cell culture comprising a protein of interest; (ii) lysing the cell culture to obtain a cell lysate; (iii) subjecting the cell lysate to a filtration column, e.g., mixing the cell lysate with a resin, such as DE52 resin and adding the resin/lysate mixture to an empty column (e.g., with a frit but without a significant amount of resin) and allowing a soluble fraction of the lysate to pass through the column to produce a clarified lysate; (iv) subjecting the clarified lysate to further fractionation by chromatography, including (a) ion exchange chromatography, e.g., by passing the clarified lysate through a column containing an ion exchange resin, e.g., an anion exchange resin such as DE52, to obtain a first eluate protein solution (ion exchange flow through); (b) subjecting the first eluate protein solution to affinity chromatography, e.g., an IMAC resin such as Ni—NTA, to obtain a second eluate protein solution; and (c) subjecting the second eluate protein solution to a desalt column to obtain a desalted protein solution; and (v) concentrating the desalted protein solution to produce a purified preparation of the protein of interest.

One or more steps of the purification method can be performed in the presence of protease inhibitors, e.g., benzamidine and phenyl methyl sulfonyl fluoride (PMSF).

Further information regarding purification of polypeptides is provided, e.g., in Robert K. Scopes, Protein Purification, Principles and Practice, Third Ed., Springer Verlag New York, 1993.

6. Determination of Protein Purity

The invention provides purified polypeptides, e.g., polypeptides that are substantially free of other cellular material, e.g., proteins. The term “substantially pure or purified preparation of a polypeptide” refers to a preparation of the polypeptides having less than about 20% (by dry weight) contaminating cellular material, e.g., nucleic acids, proteins, and lipids, and preferably having less than about 5% contaminating cellular material. Preferred preparations of the subject fusion polypeptide have less than about 2% contaminating cellular material; even more preferably less than about 1% contaminating cellular material and most preferably less than about 0.5; 0.2; 0.1; 0.01; 0.001% contaminating cellular material.

Polypeptide preparations that are “substantially free of other cellular proteins” (also referred to herein as “contaminating proteins”) refer to preparations of polypeptides having less than about 20% (by dry weight) contaminating protein, and preferably having less than about 5% contaminating protein. Preferred preparations of the subject polypeptides have less than about 2% contaminating protein; even more preferably less than about 1% contaminating protein and most preferably less than about 0.5; 0.2; 0.1; 0.01; 0.001% contaminating proteins.

The purity of the polypeptide preparation of the invention can be determined by various methods. A preferred method for determining the amount of contaminating proteins in a polypeptide preparation comprises subjecting the polypeptide preparation to gel electrophoresis, e.g., polyacrylamide electrophoresis, in the presence of specific amounts of molecular markers, and staining the gel after the electrophoresis with a protein dye. A comparison of the intensity of the band of the subject polypeptide with the molecular markers indicates the purity of the subject polypeptide preparation. Other methods for determining the amount of contaminating proteins include mass spectrometry, gel filtration and peptide sequencing according to methods known in the art.

A preferred method for determining the amount of contaminating cellular material in a polypeptide preparation comprises gel electrophoresis and silver staining of the gel.

Other methods for determining the purity of a polypeptide preparation include mass spectrometry according to methods known in the art. Yet other measurements of the purity of a polypeptide preparation include a measure of the activity of the polypeptide, as further described herein.

Protein concentrations can be determined according to the following methods: Lowry-Folin-Ciocalteau reagent; UV absorption at 280 nm (aromatic band) or 205-220 nm (peptide band); dye binding (e.g., Coomassie Blue G-250); or bis-cinchonic acid (BCA; Pierce Chemicals (Rockford, Ill.)) reagent. All of these methods are described in, e.g., Robert K. Scopes, Protein Purification, Principles and Practice, Third Ed., Springer Verlag New York, 1993, and references cited therein. Briefly, the well-known Lowry method is a relatively sensitive method giving a good color with 0.1 mg/ml or protein or less. The method using Coomassie Blue G-250 is very sensitive, fast and at least as accurate as the Lowry method. The procedure consists in mixing a polypeptide sample with the reagent and measure the blue color at 595 nm.

A preferred method for determining exact protein amounts is by dry weight determination, since it provides a suitably accurate measurement of protein amount. This can be determined, e.g., by comparison with known quantities of protein standards.

The percent recovery and degree of purity of a preparation of polypeptide can be calculated from the total amount of protein recovered after purification and the amount and/or activity of the polypeptide of interest.

7. Apparatuses

The disclosed technology enables researchers and other interested parties to automatically produce purified cell components, e.g., nucleic acids, proteins, peptides, lipids, polysaccharides, and combinations thereof, from cells in an amount sufficient to allow further characterization or experimentation with such such cell components. High-throughput production of targeted cell components can be realized by automated process control systems that use software application programs to instruct one or more processors, controllers, and actuators to control and/or monitor the operations of separation, purification, and concentration modules in such systems. Although the following systems and methods are described as being applied primarily to the purification of proteins, those skilled in the art will appreciate that the disclosed systems and methods can be used to purify and concentrate substantially any type of cell component.

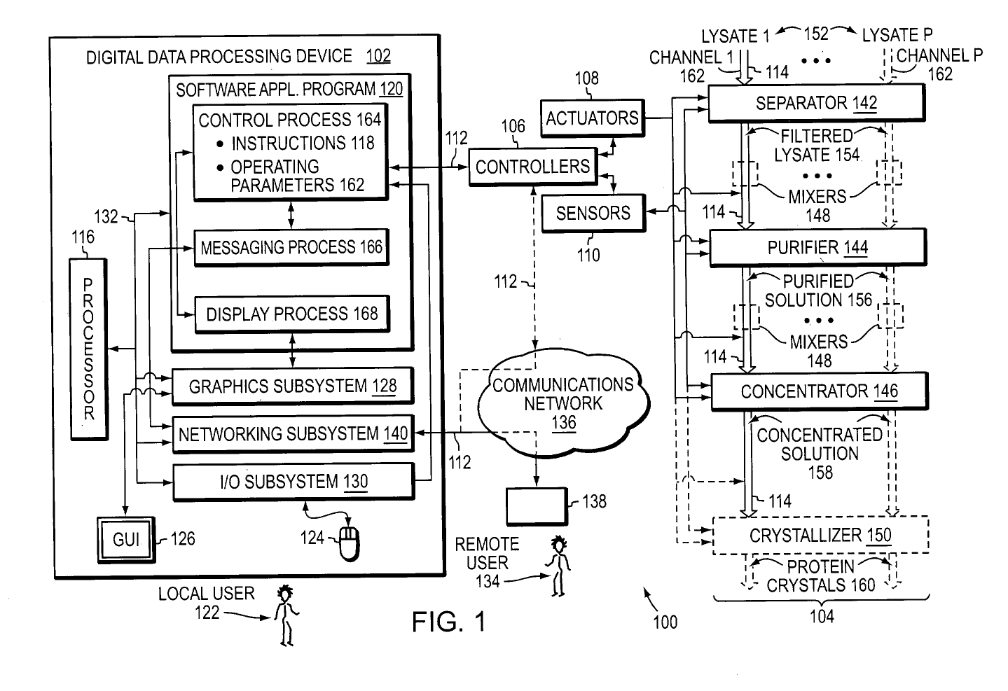

In one illustrative embodiment and with reference to FIG. 1, an automated process control system 100 capable of supporting high-throughput protein production includes a digital data processing device 102, a biochemical subsystem 104, and various controllers 106, actuators 108, sensors 110, electrical communication lines 112, and fluidic transfer lines 114 that enable the digital data processing device 102 to control and monitor the operations of the biochemical subsystem 104 during a protein purification and concentration process.

The digital data processing device 102 can be a personal computer, computer workstation (e.g., Sun, HP), laptop computer, server computer, mainframe computer, handheld device (e.g., personal digital assistant, Pocket PC, cellular telephone, etc.), information appliance, programmable logic controller, or any other type of generic or special-purpose, processor-controlled device capable of receiving, processing, and/or transmitting digital data. The digital data processing device 102 can include interface circuitry, such as digital and/or analog input/output circuits, to communicate with the actuators 108 (e.g., pumps) and sensors 110. A processor 116 refers to the logic circuitry that responds to and processes instructions 118 that drive digital data processing devices and can include, without limitation, a central processing unit, an arithmetic logic unit, an application specific integrated circuit, a task engine, and/or any combinations, arrangements, or multiples thereof.

The instructions 118 executed by the processor 116 represent, at the lowest level, a sequence of “0's” and “1's” that describe one or more physical operations of the digital data processing device 102. These instructions 118 can be pre-loaded into a programmable memory (not shown) (e.g., EEPROM) that is accessible to the processor 116 and/or can be dynamically loaded into/from one or more volatile (e.g., RAM, cache, etc.) and/or non-volatile (e.g., hard drive, etc.) memory elements communicatively coupled to the processor 116. The instructions 118 can, for example, correspond to the initialization of hardware within the digital data processing device 102, an operating system (not shown) that enables the hardware elements to communicate with each other under software control and enables other computer programs to communicate with each other, and/or software application programs 120 that are designed to perform particular functions for a user or other computer programs, such as functions relating to the purification and concentration of proteins.

A local user 122 can interact with a digital data processing device 102 by, for example, viewing a command line, graphical, and/or other user interface type and entering commands via an input device 124, such as a mouse, keyboard, touch sensitive screen, track ball, keypad, etc. The user interface 126 can be generated by a graphics subsystem 128 of the digital data processing device 102, which renders the interface into an on or off-screen surface (e.g., in a video memory and/or on a display screen). Inputs from the user 122 can be received via an input/output subsystem 130 and routed to the processor 116 via an internal bus 132 (e.g., system bus) for execution under the control of the operating system.

Similarly, a remote user 134 can interact with the digital data processing device 102 over a data communications network 136. The inputs from the remote user 134 can be received and processed in whole or in part by a remote digital data processing device 138 collocated with the remote user 134. Alternatively or in combination, the inputs can be transmitted back to and processed by the local digital data processing device 102 or to another digital data processing device via one or more networks using, for example, thin client technology (such as that developed by Citrix Systems, Inc. of Fort Lauderdale, Fla.). The user interface 126 of the local digital data processing device 102 can also be reproduced, in whole or in part, at the remote digital data processing device 138 collocated with the remote user 134 by transmitting graphics information to the remote device 138 and instructing the graphics subsystem of the remote device to render and display at least part of the interface 126 to the remote user 134. Network communications between two or more digital data processing devices typically require a network subsystem 140 (e.g., as embodied in a network interface card) to establish the communications link between the devices.

Data communications networks can comprise a series of network nodes (e.g., the local and remote digital data processing devices) that can be interconnected by network devices and communication lines 112 (e.g., public carrier lines, private lines, satellite lines, etc.) that enable the network nodes to communicate. The transfer of data (e.g., packets) between network nodes can be facilitated by network devices, such as routers, switches, multiplexers, bridges, gateways, etc., that can manipulate and/or route data from a source node to a destination node regardless of any dissimilarities in the network topology (e.g., bus, star, token ring), spatial distance (local, metropolitan, or wide area network), transmission technology (e.g., TCP/IP, Systems Network Architecture), data type (e.g., data, voice, video, or multimedia), nature of connection (e.g., switched, non-switched, dial-up, dedicated, or virtual), and/or physical link (e.g., optical fiber, coaxial cable, twisted pair, wireless, etc.) between the source and destination network nodes.

With continuing reference to FIG. 1, the exemplary biochemical subsystem 104 can include, for example, a combination of containers (e.g., bottles, chromatography columns, pressurized canisters) and/or container assemblies, chemicals, gases (e.g., nitrogen), one or more separators 142, one or more purifiers 144, one or more concentrators 146, one or more mixers 148, and/or one or more crystallizers 150, partly or entirely interconnected with flow adapters, fluidic transfer lines 114 (e.g., high purity PFA tubing, FEP tubing, etc.), gas lines, actuators 108 (e.g., single or multi-position valves, regulators, pumps (such as a mini-peristaltic pump), pressure transducers, etc.) and/or any other elements desired in automating the purification and concentration processes of proteins. The term “pump” is defined broadly to include any type of device or combination of devices capable of moving/transferring fluid, e.g., by pressure, vacuum, or otherwise.

In one illustrative embodiment, the separator 142 includes a container assembly holding one or more chromatography columns used in filtering (e.g., gravity or vacuum filtration) and/or centrifuging a crude cell lysate so as to separate out a portion of the biomass (e.g., unlysed cells, insoluble cellular debris, etc.) from the lysate (corresponding to, for example, a clarified lysate or filtered lysate 154). In one embodiment, the filtered lysate is formed by mixing a crude cell lysate with ion exchange resin, applying it to an empty column with a frit, and allowing the lysate to pass through the column. The filtered lysate 154 can then be conveyed to the purifier 144 (which can include a container assembly holding one or more chromatography columns comprising a desired chromatography resin or membrane) where a purified solution 156, having substantially pure protein of interest in a buffer solution, is formed from the filtered lysate 154. The protein solution 156 can be concentrated by the concentrator 146 (which can include a container assembly holding one or more chromatography columns used in filtering and/or centrifuging) into a concentrated solution 158 (exhibiting, for example, a purity exceeding 95%) that can be readily used for further downstream processing, such as for example, structural or biochemical studies of the protein (e.g., crystallization of the protein into crystals 160 using the crystallizer 150). One or more mixers 148 can also be interspersed between the separator, purifier, concentrator, and/or crystallizer so that various chemicals (e.g., buffers, enzymes, chemicals, etc.) can be introduced into the separation, purification, concentration, and/or crystallization processes.

The various elements of the biochemical subsystem 104 described above can be arranged to provide a plurality of channels 162 (e.g., 6 channels), where each channel 162 includes the appropriate elements for separating, purifying, concentrating, and/or crystallizing a particular protein of interest from a crude cell lysate 152. Each channel 162 can also share at least some of the controllers 106, actuators 108, and/or sensors 110 with other channels. The plurality of channels enable the automated process control system 100 to concurrently process multiple crude cell lysates 162 (which can be substantially the same or different lysates) in parallel, resulting in, for example, different purified proteins from the same lysate type, greater volume of the same purified protein from either the same or different cell lysate type, different purified proteins from different cell lysate types, etc. Further, the parallel processing of multiple cell lysates 114 can be staggered in time such that the separation, purification, and/or concentration of some or all of the lysates 114 begin at different times, so at to take advantage of the natural time delays associated with particular chemical reactions and to more efficiently utilize the resources (compute cycles, buffers, etc.) of the automated process control system 100.

The channels and chemical reactions occurring within the biochemical subsystem 104 can be configured, controlled, and monitored, at least in part, by the software application program 120. The instructions 118 of the application program 120 are executed by the processor 116 under the control of the operating system of the digital data processing device 102, resulting in one or more signals being generated by the processor (or an element under the control of the processor). These processor signals are communicated over the electrical communication lines 112 (e.g., internal or external bus, network link, etc.) to one or more controllers 106 (e.g., pump on/off/flow-rate control, valve open/close/position control, etc.), which control the operation/configuration of corresponding actuators 108 that affect the flow, operation, and interaction of the elements, fluids, and/or gases of the biochemical subsystem 104. Although the controllers 106 are shown as being independent elements of the automated process control system 100, the controllers 106 can be partly or entirely integrated into the actuators 108 (i.e., resulting in “smart” actuators) and/or the digital data processing device 102. The controllers 106 can also include internal processors that independently control, in part, the operation of the biochemical subsystem 104.

The operation of the elements within the biochemical subsystem 104 (e.g., separator 142, purifier 144, concentrator 146) can be monitored by sensors 110 (e.g., pressure sensors/transducers, ultrasound level sensors, capacitance level sensors, photoelectric level sensors, ultraviolet sensors, molecular weight sensors, PH sensors, temperature sensors, humidity sensors, etc.) that generate signals back to the processor 116 via the controllers 106 that are indicative of operational parameters (e.g., level of fluids, gas pressure, protein concentration, temperature, humidity etc.) of such operations. The signals generated by the sensors 110 are processed by the processor 116 pursuant to the program instructions 118 so as to ensure that the automated processing of the targeted protein proceeds properly, that errors are resolved and/or reported to the local or remote users 122, 134, and/or the operation of the biochemical subsystem 104 is maintained within normal/safe parameters.

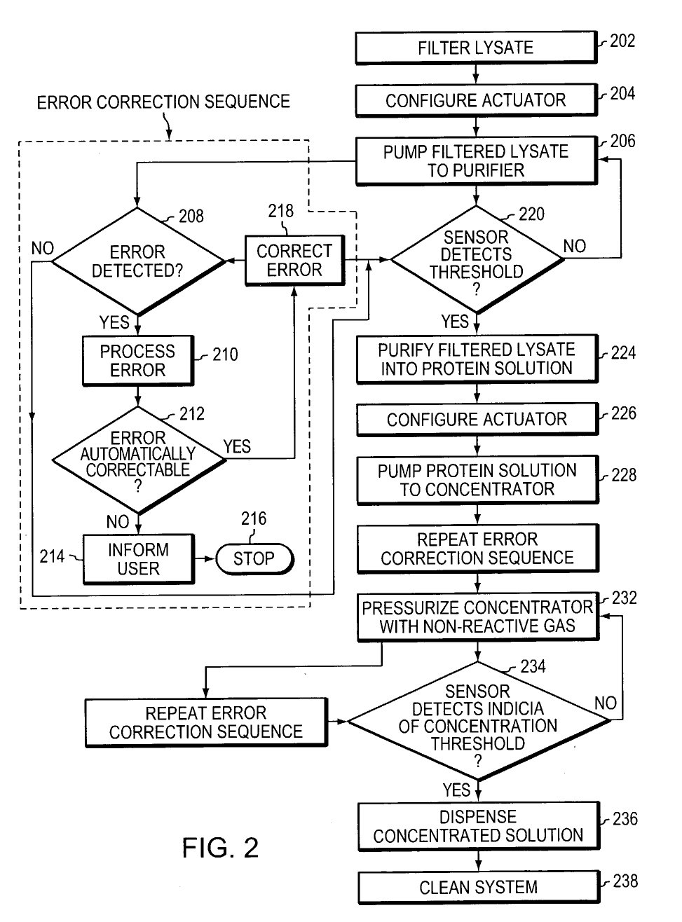

In one illustrative operation and with reference to FIGS. 1 and 2, a control process 164 of the software application program 120, executing within an execution environment (which includes, for example, instructions 118, operating parameters 162, variables, constants, software libraries, and/or other elements needed for the proper execution of the control process 164) in a memory of the digital data processing device 102, causes the processor 116 to signal the appropriate controllers 106 to configure corresponding actuators 108 so that the crude cell lysate 152 is filtered by the separator 142 (202). Sensors 110 coupled to the separator 142 monitor the progress of the filtration and signal the control process 164 via the processor 116 and controller 106, when a particular threshold is reached. In response to the signal from the sensor 110, the control process 164 instructs a controller 106 to configure an actuator 108 (204), such as a valve positioned between the separator 142 and purifier 144, so that the filtered lysate 154 can flow along the fluidic transfer line 114 between the separator 142 and purifier 144. The control process 164 then pumps the filtered lysate 154 to the purifier 144 (206).

During and/or after the pump is operated, the control process 164 determines whether an error condition has been reported by the sensors 110 (208). If an error condition has been reported, the control process 164 and the processor 116 process the error (210) and determine whether the error is of the type that is correctable (212) without input from the local or remote user 122, 134. If the error is not correctable, then the control process 164 instructs a messaging process 166 (e.g., a process interfacing with messaging software and/or hardware of the digital data processing device 102, such as electronic mail, facsimile, telephone, etc.) of the software application program 120 to inform the user 122, 134 of the error condition (214). The control process 164 can then automatically terminate the purification and concentration of the target protein (216) or await further instructions from the user. If the control process 164 deems the error condition to be correctable, then the control process 164, in concert with the processor 116, controllers 106, and/or actuators 108, attempts to correct the error (218) by, for example, changing/retrying particular valve configurations, turning the pump on and flowing a buffer or wash solution through a potentially clogged fluidic transfer line 114, etc. This automatic error recovery process can be repeated a number of times before reporting the failure of correcting the error to the user 122, 134. Once the error condition is resolved or if no error condition was previously detected, the pumping of the filtered lysate 154 continues until a sensor 110 detects that a particular threshold has been reached and signals the control process 164 accordingly (220).

The control process 164 can then instruct the purifier 144 to purify the filtered lysate 154 into a substantially purified protein solution 156 (224). The control process 164 can subsequently configure another actuator 108, such as a valve and/or a pump, to enable the purified solution 156 to flow along the fluidic transfer line 114 between the purifier 144 and the concentrator 146 (226). A pump can be instructed to pump the purified solution 156 to the concentrator 146 (228) or the purified solution 156 can be conveyed via gravity or any other mechanism to the concentrator 146. As discussed above, the automatic error detection and recovery sequence discussed above (208-218) can be repeated to ensure that the biochemical subsystem 104 is ready to concentrate the purified solution 156. Assuming that no error condition has been reported by the sensors 110 or that an error was reported and automatically resolved, the control process 164 instructs a controller 106 to configure a high pressure valve and/or other actuator 108 to pressurize the concentrator 146 with a non-reactive gas, such as nitrogen (232). The control process 164 repeats the above-mentioned error correction sequence to ensure that the pressurized concentrator 146 and associated components are properly functioning. Assuming that no errors are encountered or that errors have been corrected, a sensor 110 detects indicia of a concentration threshold of the purified solution 156 to determine whether the purified solution 156 has been successfully concentrated into a desired concentrated solution 158 (234). If concentration has been successful, the control process 164 can instruct the concentrator 146 and/or related actuator 108 to dispense the concentrated solution 158 into a holding container (236), transfer the solution 158 to the crystallizer 150, and/or otherwise remove the concentrated solution 158 from the concentrator 146 so that an automatic cleaning process can be performed to cleanse the biochemical subsystem 104 in preparation for future protein purifications (238).

A visual representation of the operational parameters 162 and/or other information relating to the operation of the automated process control system 100 can be displayed to the user 122, 134 via a display process 168 of the software application program 120. In one embodiment, the display process 168 accesses the operational parameters 162 along with other status/performance information and instructs the graphics subsystem to render and display this information in a graphical user interface 126, in accordance with a predefined format. As previously described, the GUI 126 can be displayed locally for a local user 122 or the GUI information can be transmitted to and displayed on the digital data processing device 138 of a remote user 134, via the network subsystem 140 and communications network 136.

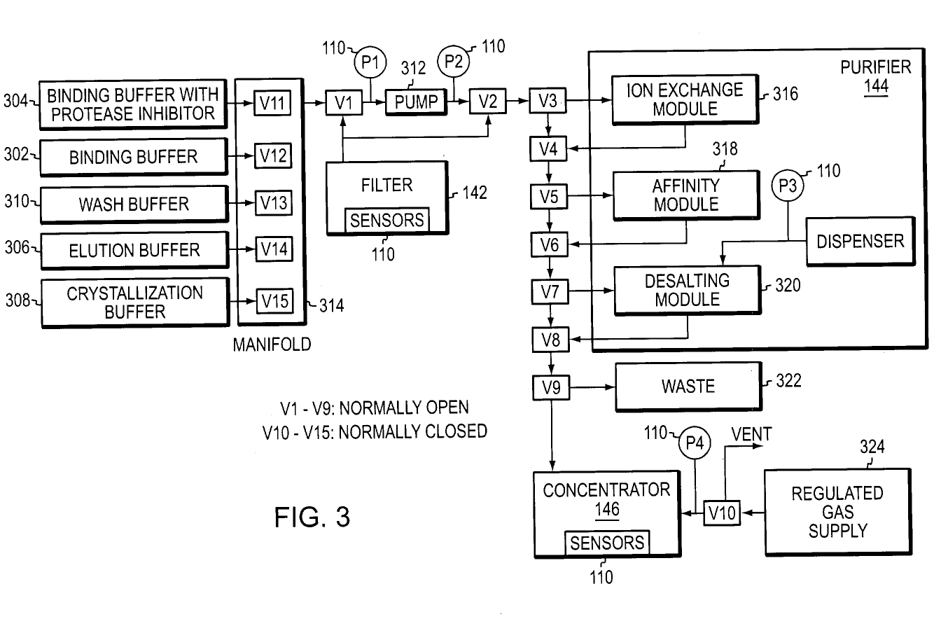

In one illustrative embodiment and with reference to FIG. 3, a channel 162 (FIG. 1) of the biochemical subsystem 104 includes a particular arrangement of actuators 108, sensors 110, fluidic/gas transfer lines 114, and buffer containers that are coupled to the filter 142, purifier 144, and concentrator 146 to enable the purification and concentration of a target protein. Various buffers, such as a binding buffer 302, a binding buffer with a protease inhibitor 304, an elution buffer 306, a crystallization buffer 308, and/or a wash buffer 310 can be introduced into the operation of the biochemical subsystem 104 in order to support/enable the reactions occurring within the filter 142, purifier 144, and/or concentrator 146 and to preload and/or wash selected elements of the biochemical subsystem 104. Each of the buffers 302-310 can be selectively coupled to the filter 142, purifier 144, and/or concentrator 146 via an arrangement of valves, fluidic transfer lines, and a pump 312. For example, a multi-position valve manifold 314 comprising a plurality of valves (V11-V15) can enable each of the buffers 304-310 to enter fluidic transfer lines 114 that can access many parts of the biochemical subsystem 104.

Various flow and/or pressure valves coupled to the fluidic transfer lines can be configured to support and/or bypass the operations of the filter 142, purifier 144, concentrator 146, and/or elements thereof. For example, the configuration of valves V1 and V2 can affect the operation of the filter 142 and any other downstream elements (e.g., the purifier 144, concentrator 146), the configuration of valves V3 and V4 can affect the operation of a ion exchange module 316 within the purifier 144, valves V5 and V6 can affect the operation of an affinity module 318 within the purifier 144, valves V7 and V8 can affect the operation of a desalting module 320 within the purifier 144, and valves V9 and V10 can affect the operation of the concentrator 146. These exemplary valves can be configured by corresponding controllers 106 (FIG. 1) under the control of the control process 164 of the software application program 120 to selectively transfer particular buffers 302-310, the filtered lysate 154, the purified solution 156, and/or the concentrated solution 158 into particular section of the biochemical subsystem 104. Sensors 110 coupled to the filter 142 and concentrator 146 provide feedback information to the control process 164 indicative of operations affecting those elements, while pressure transducers/sensors, P1-P4, monitor fluid and/or gas pressure levels to ensure the proper operation of the biochemical subsystem 104.

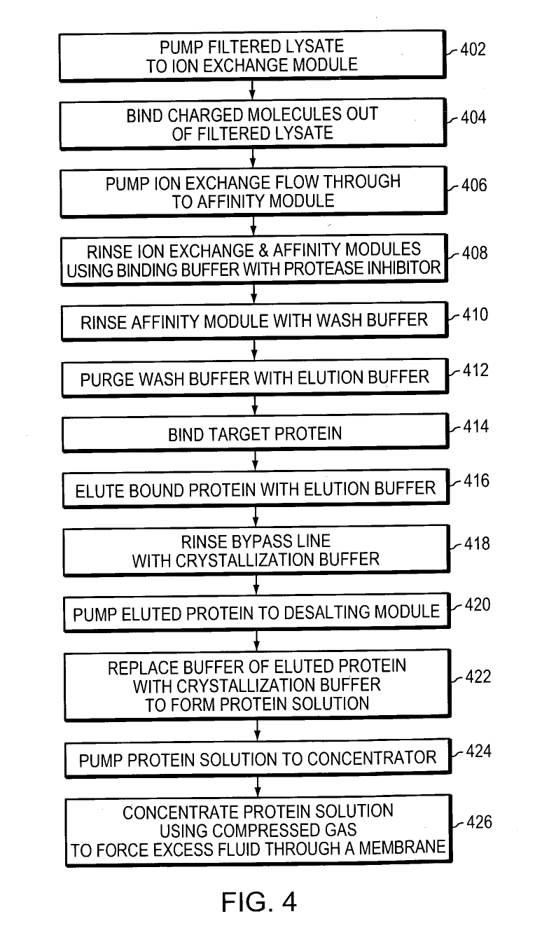

In operation and with reference to FIGS. 3 and 4, the pump 312 pumps filtered lysate 154 from the filter 142 to the ion exchange module 316 (402). The ion exchange module 316 contains, at least in part, a resin or membrane exhibiting charged molecules that bind to at least a portion of charged molecules (for example, nucleic acids) in the filtered lysate 154 (404), thereby enabling a further fractionated lysate solution (ion exchange flow through) to pass through the ion exchange module 316 and be pumped to the affinity module 318 (406). The pump 312 pumps the binding buffer with protease inhibitor 304 to the ion exchange and affinity modules 316, 318 in order to rinse these modules (408). The pump 312 then pumps the wash buffer 310 to rinse the affinity module 318 and that section of the fluidic transfer line that bypasses the affinity module (410), excess buffer 310 can be pumped to a waste module 322 for later disposal. The pump 312 can purge the bypass line filled with the wash buffer 310 using the elution buffer 306 (412).

The affinity module 318 includes a resin or membrane functionalized with a compound, such as nickel, for binding to the target protein via the affinity tag. Accordingly, when the ion exchange flow through is pumped to the affinity module 318, the target protein is bound to the resin (414), enabling unbound proteins in the solution to be removed and pumped to the waste module 322. The pump 312 can then pump the elution buffer 306 to the affinity module 318 so that the bound protein can be eluted off the column to form a protein solution (416). The desalting module 320 and the bypass line between valves V5 and V6 can be rinsed with the crystallization buffer 308 in order to prepare for the transfer of the eluted protein solution (418). The pump 312 pumps the eluted protein to the desalting module 320 (420) where gel filtration is used to further fractionate the sample and/or replace the buffer of the eluted protein with the crystallization buffer 308 to form the purified protein solution 156 (FIG. 1) (422). In one embodiment, purification involves a combination of an ion exchange column (e.g., an anion exchange column, such as DE52), an affinity column (e.g., an IMAC column, such as Ni—NTA), and a gel filtration column (e.g., a desalt column, such as Sephadex G25). One or more additional desalting/gel-filtration modules (not shown) can also be provided to further purify the protein solution 156. Gel filtration can separate different size proteins and/or protein aggregates, resulting in a purity that can exceed 99%, which can be desirable in protein crystallization processes. The purified protein solution 156 can then be pumped to the concentrator 146 (424), which uses a compressed gas, e.g., nitrogen, from a regulated gas supply 324 to force excess fluid through a membrane, until the remaining concentrated solution 158 in the concentrator 146 reaches a particular concentration and/or volume, as detected and reported to the control process 164 by the sensor 110 monitoring the operation of the concentrator 146 (426). The purified protein solution 156 can also be concentrated manually and/or semi-automatically using, for example, centrifugation.

In one illustrative embodiment, the automated process control system 100 is configured to be a modular, self-contained, stand alone, multi-channel column chromatography system for preparative scale automated protein purification and concentration. A biochemical subsystem 104 of the process control system 100 comprises a plurality of chromatography columns linked together by tubing 114 and valves 108 and uses, for example, a miniature peristaltic pump 312 to drive the purification/concentration process. Those skilled in the art will appreciate that one or more pumps can be provided for each channel, or one or more pumps can be shared across a plurality of channels. Movement of liquid within particular channels can be independently controlled, even when a pump is shared among channels, due to valve action. The system 100 processes cell lysate (crude or clarified) into purified (e.g., >95% purity), concentrated proteins that can be subjected to further downstream processing (e.g., crystallization trials) without further purification.

The biochemical subsystem 104 can have, for example, 6 independently-controlled channels allowing 6 different purification protocols to run simultaneously. Each channel can have four columns that may be changed to provide a desired column volume or resin type. In one embodiment, the purification protocols supported by the biochemical subsystem 104 can involve any combination of 4 columns and 5 buffers that enable, for example, affinity, ion exchange, hydrophobic interaction, reverse phase and/or size exclusion (desalting) chromatography. An exemplary embodiment of the process control system 100 can be designed to purify 6×HIS-Tagged proteins, although different protein tags or untagged proteins may also be purified.

In one illustrative embodiment, the environmental operating conditions (e.g., temperature, humidity, etc.) of all or part of the automated process control system 100 can be maintained within desired environmental parameters (e.g., within 0.5 degrees Celsius between 0 to 40 degrees Celsius, and humidity below 60%). For example, the biochemical subsystem 104 and/or other portions of the process control system 100 can be located within an environmentally-controlled room. Alternatively or in combination, a chassis containing the subsystem 104 and/or other portions of the process control system 100 can be environmentally sealed to maintain desired environmental conditions within the chassis that is independent from the environmental conditions outside of the chassis. In another embodiment, particular elements, such as the separator 142, purifier 144, concentrator 146, fluid transfer lines 114, and/or any related actuators, of the biochemical subsystem 104 can be separately and/or jointly housed within sub-chasses whose environmental conditions are independently controlled.

In one illustrative embodiment, the process control system 100 can automatically equilibrate all the resins (e.g., DE-52, Ni—NTA and G25 (desalt) resins) used in the biochemical subsystem 104. The process control system 100 can automatically or semi-automatically purify proteins using a variety of purification methods. The system can also prompt the user with instructions during the chromatography process.

After elution, the protein is desalted (e.g., impurities such as imidazole, glycerol and TCEP are trapped and removed) and sent to the concentrator 146. The concentrator 116 can be designed to hold 6 ultra-free concentrator units. A laser beam generated by a photoelectric level sensor (“photoeye”) can be used to automatically detect a threshold level (level at which concentration should stop) in the volume of the ultra-free cell and to stop purification/concentration in response thereto. Each concentrator unit is retractable and can include a Teflon eluate reservoir attached to a stainless steel cap and aluminum base. The process control system 100 can also automatically regenerate the desalt resin (G25 Sephadex) during the concentration process and can reuse the regenerated resin for approximately another 10 purification cycles without replacement.

The control process 164 in concert with the controllers 106 and actuators 108 interacting with the biochemical subsystem 104 can enable the biochemical subsystem 104 to automatically or semi-automatically cleanse itself by performing rinse or disinfection procedures. The rinse procedure rinses the entire biochemical subsystem 104 with dH₂O, whereas the disinfection procedure cleans the subsystem 104 with 70% ethanol and then rinses the system with dH₂O. The cleaning sequence can use cleaning racks, which are sets of columns and flow adapters similar to the columns used during normal operation, when cleaning the subsystem 104. The cleaning racks can replace the chromatography columns during the cleaning procedure. The system can also prompt the user with instructions during the cleaning procedure.

Although the process control system 100 can alert a user with e-mail notices in response to messages and/or system errors, the system 100 preferably attempts to automatically recover from these errors before informing the user of a persistent or critical error to which it is unable to recover. For example, the system 100 can include an integrated, self-declogging mechanism that attempts to automatically clear a plugged filtration column. The user can also monitor and control the process control system 100 from a location that is remote to the system 100 using, for example, a VNC viewer.

In more detail and with respect to an exemplary embodiment of the multi-channel, biochemical subsystem 104, six glass chromatography columns can be used for the purification columns. The six glass columns can use 20 μm frits and can be clamped together in a 2×3 column assembly configuration (one glass column per channel), where column assemblies are interchangeable.

Chemical mixing columns (e.g., a mixing module) 148 may be inserted before and/or after one or more of the chromatography columns to provide a reservoir for each channel where various chemicals, such as EDTA and cutting enzymes, can be added. When using cutting enzymes in the mixing column, the resulting proteins and cut tags may be washed through a second affinity column 318 (for example, Ni—NTA) where the tags bind to the column and the proteins are washed off and sent back to the mixing column 148. EDTA may then be added to the mixing column 148 and the proteins are subsequently washed through the desalt column 320 and then to the concentrator 146.

The filter columns 316 can be BioRad's Econo-Column Chromatography Column, 5.0 cm×20 cm (Item #737-5021) exhibiting a total volume of 393 ml, supporting a resin volume of cell lysate (300 mL) premixed with 10 g of DE52 resin, including an ultrasonic level sensor (Banner #Q45UR3LIU64CQ6KS), and operating at atmospheric pressure (closed system optional). Ion exchange membranes, such as Mustang Q from Pall Biopharmaceuticals, Inc., may also be used.

The DE52 column 316 (ion exchange) can be BioRad's Econo-Column Chromatography Column, 2.5 cm×10 cm (Item #737-2511) exhibiting a total volume of 49 mL, supporting a DE52—Whatman resin (WC40570-200) at a volume of 25 mL (10 g resin in 25 mL 2.5 M NaCl), supporting a void volume of approximately 8 mL, operating at a maximum operating pressure of 10 psig, providing a closed system, and using a modified BioRad's 2.5 cm Econo column flow adaptor (Item #738-0017). Ion exchange membranes, such as Mustang Q from Pall Biopharmaceuticals, Inc., may also be used.

The protein-binding column 318 (affinity) can be BioRad's Econo-Column Chromatography Column, 1.5 cm×5 cm (Item #737-1506) exhibiting a total volume of 9 mL, supporting a Superflow Ni—NTA—Qiagen (30450) or Talon Cellthru Cobalt resin—Clonetech (8910-2) at a volume of 8 mL, supporting a void volume of approximately 2 mL, operating at a maximum operating pressure of 140 psig., providing a closed system, and using a modified BioRad 1.5 cm Econo column flow adaptor (Item #738-0016). Affinity chromatography membranes may also be used.

The desalt column 320 (gel filtration) can be BioRad's Econo-Column Chromatography Column, 2.5 cm×30 cm (Item #737-2531) exhibiting a total volume of 147 mL, supporting a Sephadex G25 Fine-Amersham Pharmacia Biotech AB (17-0032-02) resin (exclusion range 1 to 5 Kda) at a volume of 100 mL (20 g of resin in 100 mL of crystallization buffer), supporting a void volume of approximately 44 mL (determined with blue dextran), operating at a maximum resin operating pressure of >145 psig., providing a closed system, and using a modified BioRad 2.5 cm Econo column flow adaptor (Item #738-0017). Membranes for size fractionation may also be used.

Proteins are washed through the desalting column 320 and sent directly to the concentrator 146, which uses a pressurized gas to concentrate the protein. The user/operator can select from different MWCOs as well as concentration levels for each independent channel.

The concentrator 146 can be an UltraFree—0.5 Centrifugal Filter Device-Millipore (5,000 MWCO or 10,000 MWCO) (UFV2BGC40) exhibiting an operation pressure of 60 psig., a maximum operating volume of approximately. 90 psig, a final volume of approximately 250 μL to >1 mL, and including a fiber-optic level sensor (Banner D10DFPQ). Proteins are concentrated by pressurizing a chamber with nitrogen (N2 is used to prevent any oxidation of the proteins). The pressure is regulated using a gas regulator. The concentrator 146 can be equipped with a laser level detection mechanism which allows the user to choose the degree of purification.

The pump 312 can be an Instech P625/66.133 miniature peristaltic pump using Tygon tube sets for pump (Reorder No. TS/P625/S5.059T), exhibiting an operating flow rate of 0 to 5 mL/min. Each of the 6 channels may have its own peristaltic pump to allow the system to run six different protocols at one time. The mini-peristaltic pumps that drive the process can be co-located with the valve packs that contain the valves.

The pressure transducers P1 and P2 for the channels can be Omega PX-26-005DV (5 psig differential), while the pressure transducer P3 for the EDTA micro-dispenser 326 can be Omega PX-26-015DV (15 psig differential) and the pressure transducer P4 for the concentrator 146 can be Omega PX-26-100DV (100 psig differential). The pressure transducers can also be located on the valve packs and are used to detect any problems with pressure in the system and to protect the user and the resins. If any of the pressures exceed their limits, the system 100 will generate an error and the system will stop. An error message can be generated to indicate some of the possible causes of the problem.

The flow adaptors in the Ni—NTA column 318 can be an Econo-column Flow adaptor 1.5 cm. (Bio-Rad cat #7380016), whereas the flow adapters in the desalt column 320 can be an Econo-column Flow adaptor 2.5 cm (Bio-Rad cat #7380017) and the flow adapters in the DE52 column 316 can be an Econo-column flow adaptor 2.5 cm (BioRad #738-0017).

The biochemical valves can be: ¼-28, 2 way valve (100-T2-NC-24-62-5M); ¼-28, 2 way valve (100-T2-NO-24-62-5M); ¼-28, 3 way valve (100-T3MP-24-62-5M); ¼-28, 3 way valve 90 psi (100T3-PP643); ¼-28, 5 valve manifold (089M5-PP652). The SMC Solenoid valves (24 V, 0.062 Orifice) can be: ¼-28, 3 way Liquid Valve (SMC-LVM115-5E-X23); M5 ports, with fittings to ¼″-28, 3 way valve, dry gas (SMC-VQ110-5L); SMC 6 channel valve manifold (SMC #LVM13R-5A-2-X35). The valve assemblies may be quickly and easily disconnected for fast trouble-shooting and maintenance.

The tubing of the fluidic transfer lines 114 can be a high purity PFA (Teflon) Tubing ⅛″ OD×1/16″ ID×50′ Length—Upchurch (1641L) and/or FEP Tubing (Cole Parmer-U-06406-62).

The valves and sensors can interface with an optically-isolated I/O system located in a separate electrical enclosure. This I/O system can be controlled by a Visual Basic interface with a C++ driver.

The level sensors can be Banner Q45UR Ultrasonic sensors, Plastic probe Remote Ultrasonic Sensor (RG Shelley—Q45UR3LIU64CQ6KS), and Micro Cable (RG Shelley—QDEC-512). The ultrasonic sensors may be installed on top of the filtration columns and each column may have its owned level sensor. The ultrasonic sensors may be used to monitor the level of lysate in the filtration column. Once the lysate reaches a preset level, the sensors signal the software application program 120 to stop pulling lysate and to move on to the next preprogrammed step in the protocol.

The photo-eye uses a laser beam to detect changes in optical properties and can thus be located under the concentrator units to detect the final volume during concentration. Photo-eye sensors are used as a concentration level switch. The sensors can be adjusted to any concentration level within the limits of the concentrator. Once the concentration reaches the set level, the sensors signal the software application program 120 to turn off the pressurized gas to that channel.

The EDTA system 326 is located at the back of the system and includes an air piston, a syringe, a reservoir, and 6 micro-dispense valves.

The temperature sensors monitor the temperature of the proteins during purification. The biochemical subsystem 104 can operate at temperatures ranging from 0° C. to 50° C.

A conductivity meter measures the conductivity of the proteins during purification.

A PH sensor measures the pH level of the proteins during purification.

A UV detector detects the presence of the proteins during elution. The proteins eluting from the desalt columns 320 are detected and then sent to the concentrator 146. The OD (optical density) of the proteins is determined and the concentration levels are automatically adjusted in accordance therewith.

A fraction collector isolates the molecular weights of the proteins for further analysis.

The system 100 can be controlled by a software application program 120 and an Opto 22 I/O control system. The custom designed software 120 supports automatic, semi-automatic, and manual purification procedures and provides easy to follow step-by-step instructions for running multiple purification protocols where appropriate. The graphical user interface 126 allows the user/operator to follow the status of each channel in substantially real time and displays the direction and rate of flow, as well as, which buffer solution and/or column(s) access the flow. The software can also be configured to send error or alert messages to anyone via electronic mail.

In operation, the exemplary process control system described above is operated in accordance with the exemplary methodology delineated below.

1) Air is purged from the buffer lines as follows:

a) Elution buffer line: Set pump flow rate to 5 mL/min; energize V14 (Elution buffer: 50 mM HEPES, 5% glycerol (v/v), 0.5 M NaCl, 250 mM Imidazole, 5 mM TCEP) for 3 minutes; turn pump 312 on in forward direction for 3 minutes: flow 15 mL of elution buffer 306 from buffer reservoir to waste 322.

b) Wash buffer line: Energize V13 (Wash buffer: 50 mM HEPES, 5% glycerol (v/v), 0.5 M NaCl, 30 mM Imidazole) for 3 minutes; turn pump 312 On in forward direction for 3 minutes: flow 15 mL of wash buffer 310 from buffer reservoir to waste 322.

c) Crystallization buffer line: Energize V15 (Crystallization buffer: 10 mM HEPES, 0.5 M NaCl) for 3 minutes; turn pump 312 On in forward direction for 3 minutes: flow 15 mL of crystallization buffer 308 from buffer reservoir to waste 322.

d) Binding buffer line: Energize V12 (Binding buffer: 50 mM HEPES, 5% glycerol (v/v), 0.5 M NaCl, 5 mM Imidazole) for 3 minutes; turn pump 312 On in forward direction for 3 minutes: flow 15 mL of binding buffer 302 from buffer reservoir to waste.

e) Binding buffer with protease inhibitors line: Energize V11 (Binding buffer with protease inhibitors: 50 mM HEPES, 5% glycerol (v/v), 0.5 M NaCl, 5 mM Imidazole, 1 mM benzamidine and 0.5 mM PMSF) for 3 minutes; turn pump 312 On in forward direction for 3 minutes: flow 15 mL of binding buffer with protease 304 from buffer reservoir to waste 322.

f) Fill-Filter Column Outlet Line: Energize V2 for 5 minutes; energize V11 (Binding buffer with protease inhibitors) for 5 minutes. Turn pump 312 On in forward direction for 5 minutes. Flows 25 ml in filtration column 142.

g) Empty-Filter Column Outlet Line: Energize V1 for 3 minutes; energize V2 for 3 minutes. Turn pump 312 On in forward direction for 3 minutes.

2) Columns are prepared as follows:

a) Preparation of Ni—NTA Columns 318. Remove the Ni—NTA column modular block and install over the Column preparation module. Gently add 8 mL Superflow Ni—NTA resin (Qiagen) Ni—NTA resin to each column. Let the resin settle and the excess buffer drip out of the column for 10 minutes. Install the Ni—NTA column block into the subsystem 104.

b) Preparation of DE52 Columns 316. Gently add 25 mL of resin to each column 316. Prepare DE52 resin (mix 60 grams of DE52 resin in 150 ml of 2.5 M NaCl) and avoid bubbles in the resin. Let the resin settle and the excess buffer drip out of the column 316 for 10 minutes. Install the column block into the subsystem 104.

c) Preparation of Desalt Columns 320. Gently add 100 mL of Sephadex G25 Fine resin (20 g in 100 mL of crystallization buffer) to each column in the Desalting Column Block. Let the resin settle and the excess buffer drip out of the column 320 for 10 minutes. Install the column block into the subsystem 104.

d) Preparation of Concentrator 146. Insert 6 labeled Ultra-free concentrator units in the concentrator system. Firmly tighten seal assembly.

3) Flow adapters are inserted in the columns blocks. The adapters should preferably be kept at their highest level in the column until the purging is completed.

4) Air is purged from the flow adapter lines as follows:

a) Purge inlet of DE52 Columns 316. Set pump flow rate to 2 mL/min. Energize V12 (Binding buffer) for 2 minutes. Turn pump 312 On in forward direction for 2 minutes. Energize V3 for 2 minutes: System pumps 4 mL of binding buffer 302 through the inlet line of each DE52 column 316 purging any air from the lines.

b) Purge inlet of Ni—NTA Columns 318. Energize V12 (Binding buffer) for 2 minutes. Energize V5 for 2 minutes. Turn pump 312 On in forward direction for 2 minutes: System pumps 4 mL of binding buffer through the inlet line of each Ni—NTA column 318 purging any air from the lines.

c) Purge before Desalt. Set pump flow rate to 5 mL/min. Energize V15 (Crystallization buffer) for 3 minutes. Turn pump 312 On in the forward direction for 3 minutes: Rinse by-pass line with 15 mL of crystallization buffer 308.

d) Purge inlet of Desalt Column 320. Set pump flow rate to 2 mL/min. Energize V15 (crystallization buffer) for 2 minutes. Energize V7 for 2 minutes. Turn pump 312 On in forward direction for 2 minutes: System pumps 4 mL of crystallization buffer 308 through the inlet line of each desalt column 320 purging any air from the lines.

5) The flow adapters are lowered and secured into all the columns blocks. The stop limit on the flow adapters should not be passed.

6) Equilibrate the columns as follows:

a) Equilibration of the DE52 Columns 316. Energize V12 (Binding buffer) for 15 minutes. Energize V3 for 15 minutes. Energize V4 for 15 minutes. Turn pump 312 On in forward direction for 15 minutes: System pumps 30 mL of binding buffer 302 over each DE52 column 316 purging any air from the system.

b) Equilibration of the Ni—NTA Columns 318. Energize V12 (Binding buffer) for 15 minutes. Energize V5 for 15 minutes. Energize V6 for 15 minutes. Turn pump 312 On in forward direction for 15 minutes: System pumps 30 mL of Binding buffer 302 over each Ni—NTA column 318 purging any air from the system.

c) Purge line with Crystallization buffer 308. Set pump flow rate to 5 mL/min. Energize V15 (Crystallization buffer) for 3 minutes. Turn pump 312 On in the forward direction for 3 minutes: Rinse by-pass line with 15 mL of crystallization buffer 308.

d) Equilibration of the Desalt Columns 320. Energize V15 (Crystallization buffer) for 30 minutes. Energize V7 for 30 minutes. Energize V8 for 30 minutes. Turn pump 312 On in forward direction for 30 minutes. System pumps 60 mL of crystallization buffer 308 over each desalt column 320 purging any air from the system.

7) Preparation of Concentrator 146:

a) Turn Photoeye Off.

b) Fill Concentrator 146. Set pump flow rate to 5 mL/min. Energize V9 for 3 minutes. Energize V15 and keep on until “Flow lysate” task. Turn pump 312 On in forward direction for 3 minutes. System pumps 15 mL of crystallization buffer 308 into concentrator units 146.

c) Empty Concentrator 146. Energize V10 for 5 minutes: Pressurize concentrator cell to 60 psig and allow most of the crystallization buffer 308 to pass through the filter membrane.

8) Preparation of Cell Lysate. A cell culture is flash frozen in liquid nitrogen and then thawed. The thawed sample is supplemented with 0.5% CHAPS and 12.5 U/ml Benzonase Nuclease. The sample is then gently rocked on a Nutator (VWR, setting 3) at room temperature for 45 minutes. Cells are then lysed by sonication (1×30 seconds, output 4 to 5, 80% duty cycle, in a Branson Sonifier, VWR) and an aliquot is saved for a gel sample.

9) Preparation of DE52 resin. Mix 60 g of DE52 resin (Whatman, anion exchanger) in 150 ml of 2.5 M NaCl for 6 protein samples. Equilibrate resin with 180 mL Binding buffer. Once resin has been equilibrated, eject resin into a 1000 cc beaker and resuspend in 600 mL binding buffer 302.

10) Preparation of lysate/DE52 slurry. Add 100 mL of resuspended DE52 to each sonicated sample (lysate). Add approximately 100 mL of binding buffer 302 to each slurry to bring the total volume to about 300 mL. Mix thoroughly and gently.

11) Gently load Cell Lysate onto Filtration Column 142.

12) Chromatography

a) Protein Binding

i) Flow Lysate. Set pump flow rate to 2 mL/min. Energize V1-bring filtration column 142 on-line. Energize V3 and V4—bring DE-52 column 316 on-line. Energize V5 and V6—bring Ni—NTA column 318 on-line. De-energize V15. Turn pump 312 On in forward direction until the ultrasonic level sensor signals that the threshold level has been reached. De-energize V3, V4, V5, V6 and V15. V1 stays on (prevents fluid hammer on pressure transducers). Turn Off pump 312.

ii) Rinse DE52 Column 316. Energize V3, V4, V5, V6 and V11 for 12 minutes: bring DE-52 and Ni—NTA columns 316, 318 on-line. De-energize V1. Turn pump 312 On in the forward direction for 12 minutes: rinse the DE52 and Ni—NTA Columns 316, 318 with 24 mL of binding buffer with Protease Inhibitor Cocktail 304.

iii) Purge line with Wash buffer 310. Set pump flow rate to 5 mL/min. Energize V13 for 3 minutes (Wash buffer). Turn pump 312 On in the forward direction for 3 minutes: Rinsing by-pass line with 15 mL of wash buffer 310.

iv) Wash. Set pump flow rate to 2 mL/min. Energize V5, V6 and V13 for 100 minutes: bring Ni—NTA column 318 on-line. Turn pump 312 On in the forward direction for 100 minutes: rinse Ni—NTA column 318 with 200 mL of wash buffer 310.

v) Purge line with Elution buffer 306. Set pump flow rate to 5 mL/min. Energize V14 for 3 minutes (Elution buffer). Turn pump 312 On in the forward direction for 3 minutes: rinse by-pass line with 15 mL of elution buffer 306.

b) Protein Elution and Desalting.

i) Elution. Set pump flow rate to 2 mL/min. Energize V14 for 20 minutes (Elution buffer). Energize V5 and V6 for 20 minutes—bring Ni—NTA column 318 on-line. Energize V7 and V8 for 20 minutes—bring Desalt column 320 on-line. Turn pump 312 On in the forward direction for 20 minutes: elute Ni—NTA column 318 with 40 mL of elution buffer 306.

ii) Purge line with Crystallization buffer 308. Set pump flow rate to 5 mL/min. Energize V15 for 3 minutes (Elution buffer). Turn pump 312 On in the forward direction for 3 minutes: rinse by-pass line with 15 mL of crystallization buffer 308.

iii) Desalting. Set pump flow rate to 2 mL/min. Energize V15 for 30 minutes (crystallization buffer). Energize V7 and V8 for 30 minutes—bring Desalt column 320 on-line. Energize V9 for 30 minutes—bring concentrator 146 on-line. Turn pump 312 On in the forward direction for 30 minutes: send eluate to Desalt with 60 mL of elution buffer 306.

iv) Supplement each sample with EDTA to a final concentration of 1 mM, wait 15 minutes, then add DTT to a final concentration of 0.2 mM. Check protein concentration using a UV spectrophotometer.

c) Protein Concentration and Desalt Regeneration.

i) Start Protein Concentration. Pressurize system with 60 psig of gaseous nitrogen. Energize V10—pressurize concentrator 146. De-energize V10 when Banner Fiber-Optic Sensors senses that the set-point level of 300 μL has been reached. When all channels are concentrated. Release the pressure off the system.

ii) If a protein has not been concentrated within 12 hours, the display will indicate “Hard to Concentrate. Click OK to continue, click Terminate to stop the process.”

iii) Measure Concentration. Put 2 uL of concentrated protein in 150 uL of 0.5M NaCl solution and read OD280 in UV Spec. Then calculate concentration (UV reading/OD 0.1% *Dilution factor (76)=mg/mL, multiply by final concentrated volume to get total mgs.)

13) System Clean-up (Full Cleaning).

a) Disinfection Option (Disinfect with 70% Ethanol and rinse with dH₂O).

i) Cleaning Preparation. Prepare 100 mL of a 70% Ethanol solution and put in Cleaning solution bottle. Transfer inlet of binding buffer with Protease Inhibitor line into the Cleaning bottle containing 70% Ethanol. Transfer inlet of Elution buffer line into the Cleaning bottle containing 300 mL of dH₂O. Remove inlet of Crystallization buffer line from the Crystallization bottle. Keep it in the air. Transfer inlet of Wash and Binding buffer lines into the WASTE bottle, being careful to not let it touch the waste.

ii) Empty filtration column 142. Set pump flow rate to 5 mL/min. Energize V1 for 5 minutes. Turn pump 312 on in the forward direction for 5 minutes.

iii) Disinfect By-Pass line. Set pump flow rate to 5 mL/min. Energize V11 (keep on). Turn pump 312 On in the forward direction for 5 minutes.

iv) Disinfect Columns. Energize V3, V4, V5, V6, V7 and V8 for 5 minutes. Turn pump 312 On in the forward direction for 5 minutes.

v) Disinfect Lysate. Energize V2 for 5 minutes. Turn pump 312 On in the forward direction for 5 minutes. Energize V1 for 5 minutes. Turn pump 312 On in the forward direction for 5 minutes.

vi) Disinfect Concentrator Line. Energize V9 for 3 minutes. Turn pump 312 On in the forward direction for 3 minutes.

vii) Empty Disinfected Concentrator. Energize V10 for 3 minutes. De-Energize V11.

viii) Rinse with water. Set pump flow rate to 5 mL/min. Energize V14 (keep on). Turn pump 312 On in the forward direction for 5 minutes.

ix) Rinse Columns. Energize V3, V4, V5, V6, V7 and V8 for 5 minutes. Turn pump 312 On in the forward direction for 5 minutes.

x) Rinse Lysate. Energize V2 for 5 minutes. Turn pump 312 On in the forward direction for 5 minutes. Energize V1 for 5 minutes. Turn pump 312 On in the forward direction for 5 minutes.

xi) Rinse Concentrator Line. Energize V9 for 3 minutes. Turn pump 312 On in the forward direction for 3 minutes.

xii) Empty Rinsed Concentrator. Energize V10 for 3 minutes. De-Energize V14.

xiii) Purge air from By-Pass Line. Set pump flow rate to 5 mL/min. Energize V15 (keep on). Turn pump 312 On in the forward direction for 5 minutes.

xiv) Purge air from Columns. Energize V3, V4, V5, V6, V7 and V8 for 5 minutes. Turn pump 312 On in the forward direction for 5 minutes.

xv) Purge air Lysate. Energize V2 for 5 minutes. Turn pump 312 On in the forward direction for 5 minutes. Energize V1 for 5 minutes. Turn pump 312 On in the forward direction for 5 minutes. De-Energize V15.

xvi) Empty Protease Line. Set pump flow rate to −5 mL/min (reverse flow). Energize V9 and V11 for 3 minutes. Turn pump 312 On in the reverse direction for 3 minutes. De-Energize V9 and V11.

xvii) Empty Elution Line. Energize V9 and V14 for 3 minutes. Turn pump 312 On in the reverse direction for 3 minutes. De-Energize V9 and V14.

xviii) Empty Binding Line. Energize V9 and V12 for 3 minutes. Turn pump 312 On in the reverse direction for 3 minutes. De-Energize V9 and V12.

xix) Empty Wash Line. Energize V9 and V13 for 3 minutes. Turn pump 312 On in the reverse direction for 3 minutes. De-Energize V9 and V13.

xx) Remove Cleaning Racks. System is ready for next purification.

b) Rinse Option (Rinse with dH₂O only).

i) Cleaning Preparation. Put inlet of Elution buffer line into the Cleaning bottle containing 300 mL of dH₂O. Remove inlet of Crystallization buffer line from the Crystallization bottle. Keep it in the air.

ii) Empty Filtration Column. Set pump flow rate to 5 mL/min. Energize V1 for 5 minutes. Turn pump 312 On in the forward direction for 5 minutes.

iii) Rinse with water. Set pump flow rate to 5 mL/min. Energize V14 (keep on). Turn pump 312 On in the forward direction for 5 minutes.

iv) Rinse Columns. Energize V3, V4, V5, V6, V7 and V8 for 5 minutes. Turn pump 312 On in the forward direction for 5 minutes.

v) Rinse Lysate. Energize V2 for 5 minutes. Turn pump 312 On in the forward direction for 5 minutes. Energize V1 for 5 minutes. Turn pump 312 On in the forward direction for 5 minutes.

vi) Rinse Concentrator Line. Energize V9 for 3 minutes. Turn pump 312 On in the forward direction for 3 minutes.

vii) Empty Rinsed Concentrator. Energize V10 for 3 minutes. De-Energize V14.

viii) Purge By-Pass Line. Set pump flow rate to 5 mL/min. Energize V15 (keep on). Turn pump 312 On in the forward direction for 5 minutes.

ix) Purge Columns. Energize V3, V4, V5, V6, V7 and V8 for 5 minutes. Turn pump 312 On in the forward direction for 5 minutes.

x) Purge Lysate. Energize V2 for 5 minutes. Turn pump 312 On in the forward direction for 5 minutes. Energize V1 for 5 minutes. Turn pump 312 On in the forward direction for 5 minutes. De-Energize V15.

xi) Empty Protease Line. Set pump flow rate to −5 mL/min (reverse flow). Energize V9 and V11 for 3 minutes. Turn pump 312 On in the reverse direction for 3 minutes. De-Energize V9 and V11.

xii) Empty Elution Line. Energize V9 and V14 for 3 minutes. Turn pump 312 On in the reverse direction for 3 minutes. De-Energize V9 and V14.

xiii) Empty Binding Line. Energize V9 and V12 for 3 minutes. Turn pump 312 On in the reverse direction for 3 minutes. De-Energize V9 and V12.

xiv) Empty Wash Line. Energize V9 and V13 for 3 minutes. Turn pump 312 On in the reverse direction for 3 minutes. De-Energize V9 and V13.

xv) Remove Cleaning Racks. System is ready for next purification.

14) Partial Cleanup (optional method of cleanup as compared to step 13). The concentrator inlet line does not get cleaned).

a) Disinfection Option (Disinfect with 70% Ethanol and rinse with dH₂O).

i) Cleaning Preparation. Prepare 100 mL of a 70% Ethanol solution and put in Cleaning solution bottle. Transfer inlet of Binding buffer with Protease Inhibitor line into the Cleaning bottle containing 70% Ethanol. Transfer inlet of Elution buffer line into the Cleaning bottle containing 300 mL of dH₂O. Remove inlet of Crystallization buffer line from the Crystallization bottle. Keep it in the air. Transfer inlet of Wash and Binding buffer lines into the waste bottle (Don't let it touch the waste).

ii) Empty Filtration Column. Set pump flow rate to 5 mL/min. Energize V1 for 5 minutes. Turn pump 312 On in the forward direction for 5 minutes.

iii) Disinfect By-Pass line. Set pump flow rate to 5 mL/min. Energize V11 (keep on). Turn pump 312 On in the forward direction for 5 minutes.

iv) Disinfect Columns. Energize V3, V4, V5, V6, V7 and V8 for 5 minutes. Turn pump 312 On in the forward direction for 5 minutes.

v) Disinfect Lysate. Energize V2 for 5 minutes. Turn pump 312 On in the forward direction for 5 minutes. Energize V1 for 5 minutes. Turn pump 312 On in the forward direction for 5 minutes.

vi) De-Energize V11.

vii) Rinse with water. Set pump flow rate to 5 mL/min. Energize V14 (keep on). Turn pump 312 On in the forward direction for 5 minutes.

viii) Rinse Columns. Energize V3, V4, V5, V6, V7 and V8 for 5 minutes. Turn pump 312 On in the forward direction for 5 minutes.

ix) Rinse Lysate. Energize V2 for 5 minutes. Turn pump 312 On in the forward direction for 5 minutes. Energize V1 for 5 minutes. Turn pump 312 On in the forward direction for 5 minutes. De-Energize V14.

x) Purge air from By-Pass Line. Set pump flow rate to 5 mL/min. Energize V15 (keep on). Turn pump 312 On in the forward direction for 5 minutes.

xi) Purge Columns. Energize V3, V4, V5, V6, V7 and V8 for 5 minutes. Turn pump 312 On in the forward direction for 5 minutes.

xii) Purge Lysate. Energize V2 for 5 minutes. Turn pump 312 On in the forward direction for 5 minutes. Energize V1 for 5 minutes. Turn pump 312 On in the forward direction for 5 minutes. De-Energize V15.

xiii) Empty Protease Line. Set pump flow rate to −5 mL/min (reverse flow). Energize V11 for 3 minutes. Turn pump 312 On in the reverse direction for 3 minutes. De-Energize V11.

xiv) Empty Elution Line. Energize V14 for 3 minutes. Turn pump 312 On in the reverse direction for 3 minutes. De-Energize V14.

xv) Empty Binding Line. Energize V12 for 3 minutes. Turn pump 312 On in the reverse direction for 3 minutes. De-Energize V12.

xvi) Empty Wash Line. Energize V13 for 3 minutes. Turn pump 312 On in the reverse direction for 3 minutes. De-Energize V13.

xvii) Remove Cleaning Racks. System is ready for next purification.

b) Rinse Option (Rinse with dH₂O only).

i) Cleaning Preparation. Put inlet of Elution buffer line into the Cleaning bottle containing 300 mL of dH₂O. Remove inlet of Crystallization buffer line from the Crystallization bottle. Keep it in the air.

ii) Empty Filtration Column. Set pump flow rate to 5 mL/min. Energize V1 for 5 minutes. Turn pump 312 On in the forward direction for 5 minutes.

iii) Rinse with water. Set pump flow rate to 5 mL/min. Energize V14 (keep on). Turn pump 312 On in the forward direction for 5 minutes.

iv) Rinse Columns. Energize V3, V4, V5, V6, V7 and V8 for 5 minutes. Turn pump 312 On in the forward direction for 5 minutes.

v) Rinse Lysate. Energize V2 for 5 minutes. Turn pump 312 On in the forward direction for 5 minutes. Energize V1 for 5 minutes. Turn pump 312 On in the forward direction for 5 minutes. De-Energize V14.

vi) Purge By-Pass Line. Set pump flow rate to 5 mL/min. Energize V15 (keep on). Turn pump 312 On in the forward direction for 5 minutes.

vii) Purge Columns. Energize V3, V4, V5, V6, V7 and V8 for 5 minutes. Turn pump 312 On in the forward direction for 5 minutes.

viii) Purge Lysate. Energize V2 for 5 minutes. Turn pump 312 On in the forward direction for 5 minutes. Energize V1 for 5 minutes. Turn pump 312 On in the forward direction for 5 minutes. De-Energize V15.

ix) Empty Protease Line. Set pump flow rate to −5 mL/min (reverse flow). Energize V1 for 3 minutes. Turn pump 312 On in the reverse direction for 3 minutes. De-Energize V11.

x) Empty Elution Line. Energize V14 for 3 minutes. Turn pump 312 On in the reverse direction for 3 minutes. De-Energize V14.

xi) Empty Binding Line. Energize V12 for 3 minutes. Turn pump 312 On in the reverse direction for 3 minutes. De-Energize V12.

xii) Empty Wash Line. Energize V13 for 3 minutes. Turn pump 312 On in the reverse direction for 3 minutes. De-Energize V13.

Remove Cleaning Racks. System is ready for next purification.

Although the present invention has been described with reference to specific details, it is not intended that such details should be regarded as limitations upon the scope of the invention, except as and to the extent that they are included in the accompanying claims. For example, although particular software processes have been provided, those skilled in the art will recognize that the functions provided by these processes can be combined with and/or dispersed among one or more different software processes. Further, the various operations of the process control system 100 can be performed locally or in a distributed fashion, for example, all or part of the control and monitoring hardware and software can be located at a plurality of local and/or remote locations relative to the biochemical subsystem 104 and associated actuators 108. The disclosed techniques used in purifying and concentrating the target protein can also be modified to occur in a different order, using different combinations of chemical reactions and compositions/buffers, etc.

Exemplification

### EXAMPLE 1

Purification of His-tagged Proteins

The present example describes purification of a protein using the methods described herein for production and clarification of a lysate in conjunction with manual protein purification over a variety of columns. These methods may also be used in conjunction with the systems and apparatuses as described herein.

An expression construct clone encoding a His-tagged polypeptide is introduced into an expression host. The resultant cell line is then grown in cultures to allow large amounts of the recombinant protein to accumulate in the cells. Various bacterial host strains may be used for protein expression, such as, BL21-Gold (DE3) supplemented with a plasmid called pUBS520, which directs expression of tRNA for arginine (agg and aga) and serves to augment the expression of the recombinant protein in the host cell (Gene, vol. 85 (1989) 109-114). The expression construct may also be transformed into BL21-Gold (DE3) without pUBS520, BL21-Gold (DE3) Codon-Plus (RIL) or (RP) (Stratagene) or Roseatta (DE3) (Novagen), the latter two of which contain genes encoding tRNAs. Alternatively, the expression construct may be transformed into BL21 STAR E. coli (Invitrogen) cells which has an Rnase deficiency that reduces degradation of recombinant mRNA transcript and therefore increases the protein yield.

Transformed cells are grown in LB medium supplemented with the appropriate antibiotics up to a final concentration of 100 μg/ml. The cultures are shaken at 37° C. until they reach an optical density (OD₆₀₀) between 0.6 and 0.7. The cultures are then induced with isopropyl-beta-D-thiogalactopyranoside (IPTG) to a final concentration of 0.5 mM at 15° C. for 10 hours, 25° C. for 4 hours, or 30° C. for 4 hours.

The cell culture is flash-frozen in liquid nitrogen, thawed and supplemented with 100 μl of protease inhibitor (0.1 M benzamidine and 0.05 M PMSF), 0.5% CHAPS, and 5 U/ml Benzonase Nuclease. The sample is then gently rocked on a Nutator (VWR, setting 3) at room temperature for 45 minutes. Cells are then lysed by sonication (1×30 seconds, output 4 to 5, 80% duty cycle, in a Branson Sonifier, VWR) and an aliquot is saved for a gel sample.

Alternatively, the cells are harvested by centrifugation and the pellets are resuspended in 25 ml HEPES buffer (50 mM, pH 7.5) and flash-frozen in liquid nitrogen. The frozen pellet is thawed and supplemented with 100 μl of protease inhibitor (0.1 M benzamidine and 0.05 M PMSF), 0.5% CHAPS, and 5 U/ml Benzonase Nuclease. Cells are then lysed by sonication (1×30 seconds, output 4 to 5, 80% duty cycle, in a Branson Sonifier, VWR) and an aliquot is saved for a gel sample.

DE52 resin for the filtration step is prepared by resuspending 10 g of DE52resin (for each 100 mL culture) in 25 mL of 2.5 M NaCl. The resin is then applied to a large Biorad Econo (5.0×30 cm×589 mL) column and equilibrated with 30 mL of binding buffer (50 mM HEPES pH 7.5, 5% glycerol (v/v), 0.5 M NaCl, 5 mM imidazole, 1 mM benzamidine and 0.5 mM PMSF). The equilibrated resin is then ejected into a beaker and resuspended in ˜50-100 mL of Binding buffer.

A second batch of DE52 resin is prepared for an ion exchange column by resuspending 10 g of DE52 resin (per each 100 mL of culture) in 25 mL of 2.5 M NaCl. The resin is then applied to a Biorad Econo (2.5×20 cm×98 mL) column and equilibrated with 30 mL of binding buffer (50 mM Hepes, 5% glycerol (v/v), 0.5 M NaCl, 5 mM imidazole, 1 mM benzamidine and 0.5 mM PMSF). For each 100 mL of culture, 8 mL of Ni—NTA resin is applied to a Biorad Econo (1.5×15 cm×27 mL) column and equilibrated with 30 mL of binding buffer.

The crude cell lysates are decanted into a beaker and mixed with the reconstituted DE52 resin. The mixture is diluted to a total volume of ˜300 mL with Binding buffer and mixed gently. 30 mL of the cell lysate-DE52 slurry is applied to a large DE52 Biorad Econo column (5.0×30 cm×589 mL) and allowed to settle for 5-10 minutes. A pump is then attached to the column and adjusted to a speed of ˜6.00. The remaining cell lysate-DE52 slurry is applied to the column in 30 mL batches. After the lysate has passed through the DE52 filtration column, the pump is turned off.

The protein sample from the DE52 filtration column is then passed over the ion exchange column comprising DE52 resin (2.5×20 cm×98 mL). Once all lysate has passed through the DE52 anion exchange column, the column is washed with 20 mL of binding buffer. Both the binding and wash steps are carried out using gravity flow.

The protein sample and wash from the DE52 ion exchange column (ion exchange flow through) is then passed over the Ni—NTA column. The column is washed with at least 200 mL of wash buffer (50 mM HEPES, 5% glycerol (v/v), 0.5 M NaCl, 30 mM imidazole). Both the binding and wash steps are carried out using gravity flow. A pump may be used (e.g., at a speed of ˜6.00-12.00) to load and/or wash the columns. His-tagged protein is eluted off the Ni—NTA column with elution buffer (50 mM HEPES, 5% glycerol (v/v), 0.5 M NaCl, 250 mM imidazole, 5 mM TCEP) until no more protein is observed in the aliquots of eluate as measured using Bradford reagent (Biorad). The eluate is supplemented with 1 mM of EDTA and 0.2 mM DTT.

The samples are assayed by SDS-PAGE and stained with Coomassie Blue, with protein purity determined by visual staining.

Two methods may be used to remove the His tag located at either the C or N-terminus of the protein, if desired. In method one, a sample of purified polypeptide are supplemented with 2.5 mM CaCl₂ and an appropriate amount of thrombin (the amount added will vary depending on the activity of the enzyme preparation) and incubated for ˜20-30 minutes on ice in order to remove the His tag. In method two, a sample of purified polypeptide is combined with thirty units of recombinant TEV protease in 50 mM TRIS HCl pH=8.0, 0.5 mM EDTA and 1 mM DTT, followed by incubation at 4° C. overnight, to remove the His tag.

The protein sample is then dialyzed in dialysis buffer (10 mM HEPES, pH 7.5, 5% glycerol (v/v) and 0.5 M NaCl) for at least 8 hours using a Slide-A-Lyzer (Pierce) appropriate for the molecular weight of the recombinant protein. An aliquot of the cleaved and dialyzed samples is then assayed by SDS-PAGE and stained with Coomassie Blue to determine the purity of the protein and the success of cleavage.

The remainder of the sample is centrifuged at ˜2700-3000 rpm at 4° C. for 10-15 minutes to remove any precipitant and supplemented with 100 μl of protease inhibitor cocktail (0.1 M benzamidine and 0.05 M PMSF) (NO Bioshop). The protein is then applied to a second Ni—NTA column (˜5-8 ml of resin on a 1.5×15 cm×27 mL column) to remove the His-tags and eluted with binding buffer or wash buffer until no more protein is eluting off the column as assayed using the Bradford reagent. The eluted sample is supplemented with 1 mM EDTA and 0.6 mM of DTT and concentrated to a final volume of ˜15 mls using a Millipore Concentrator with an appropriately sized filter at 2700 rpm at 4° C. The samples are then dialyzed overnight against crystallization buffer and concentrated to a final volume of ˜0.3-0.7 ml.

Proteins may also be concentrated using ammonium sulfate precipitation. Ammonium sulfate may be slowly added to the dialyzed protein sample with stirring to a maximum of 45% of the volume of the protein sample. The sample is then centrifuged for ˜30 minutes at 3700 rpm at 4° C. The pellet is resuspended in crystallization buffer to yield a final protein concentration of ˜40 mg/mL.

Prepared and purified in this way, the recombinant proteins are essentially the only protein visualized in the SDS-PAGE assay using Coomassie Blue described above, which is at least about 95% or greater purity.

Equivalents

The present invention provides among other things novel methods and apparatuses for purification. While specific embodiments of the subject invention have been discussed, the above specification is illustrative and not restrictive. Many variations of the invention will become apparent to those skilled in the art upon review of this specification. The appended claims are not intended to claim all such embodiments and variations, and the full scope of the invention should be determined by reference to the claims, along with their full scope of equivalents, and the specification, along with such variations.

Incorporation by Reference

All publications and patents mentioned herein are hereby incorporated by reference in their entirety as if each individual publication or patent was specifically and individually indicated to be incorporated by reference. In case of conflict, the present application, including any definitions herein, will control.

What is claimed is:

1. A method for producing a cell lysate, comprising:
(a) adding detergent to a cell culture; and
(b) sonicating the cell culture, wherein the density of the cell culture upon harvesting is not significantly increased by external manipulation prior to sonication, thereby producing a cell lysate.

2. The method of claim 1, wherein the cell culture is not centrifuged prior to sonication.

3. The method of claim 1, which further comprises freezing and thawing the cell culture before or after sonication.

4. The method of claim 1, which further comprises incubating the cell culture with the detergent for at least about 15 minutes prior to sonicating the cell culture.

5. The method of claim 1, wherein the detergent is added to the cell culture at a final concentration of about 0.1% to about 1% weight/volume of detergent.

6. The method of claim 1, wherein the detergent is one or more of the following: SDS, CHAPS, NP-40, and Triton X-100.

7. The method of claim 1, wherein the cell culture comprises cells transformed with an expression vector for the production of a recombinant protein.

8. The method of claim 1, which further comprises adding one or more of the following components before or after sonication: lysozyme, protease inhibitors, DNAse, and RNAse.

9. The method of claim 1, wherein the cell culture is a culture comprising prokaryotic cells.

10. The method of claim 1, wherein the cell culture is at least about 100 mLs.

11. The method of claim 10, wherein the cell culture is at least about 1 liter.

12. A method for clarifying a cell lysate, comprising:
(a) mixing a crude cell lysate with ion exchange resin;
(b) applying the mixture to a column; and
(c) passing the lysate through the column, thereby producing a clarified cell lysate.

13. The method of claim 12, further comprising diluting the crude cell lysate with buffer at a ratio of about 1:1 to 1:3 volume/volume (lysate:buffer).

14. The method of claim 12, wherein about 1 g of ion exchange resin (dry weight) is added for each 10 mL of cell culture used to produce the crude lysate.

15. The method of claim 12, wherein the ion exchange resin is an anion exchange resin.

16. The method of claim 15, wherein the anion exchange resin is one or more of the following: diethylaminoethyl (DEAE), quaternary aminoethyl (QAE), and quaternary amine (Q) resin.

17. The method of claim 12, wherein a peristaltic pump is used to pass the lysate through the column.

18. A method for producing a clarified cell lysate, comprising:
(a) adding detergent to a cell culture;
(b) sonicating the cell culture to produce a crude cell lysate, wherein the density of the cell culture upon harvesting is not significantly increased by external manipulation prior to sonication;
(c) mixing the crude cell lysate with ion exchange resin;
(d) applying the mixture to a column; and
(e) passing the lysate through the column, thereby producing a clarified cell lysate.

19. The method of claim 18, wherein the cell culture is not centrifuged prior to sonication.

20. A method for purifying a polypeptide, comprising: (a) obtaining a crude cell lysate; (b) mixing the crude cell lysate with ion exchange resin; (c) applying the mixture to a column and passing the lysate through the column, thereby producing a clarified cell lysate; (d) applying the clarified cell lysate to an affinity column under conditions to promote binding of polypeptides to the column resin; (e) washing the affinity column to remove non-specifically bound polypeptides; and (f) eluting the bound polypeptides from the affinity resin, thereby producing a purified polypeptide.

21. The method of claim 20, wherein the polypeptide is at least 90% pure by weight.

22. The method of claim 21, wherein the polypeptide is at least 95% pure by weight.

23. The method of claim 20, wherein the polypeptide is at least 98% pure by weight.

24. The method of claim 23, wherein the polypeptide is at least 99% pure by weight.

25. The method of claim 20, wherein the polypeptide is labeled with an isotopic label or a heavy atom.

26. The method of claim 20, which further comprises passing the clarified cell lysate over an anion exchange column prior to applying the lysate to an affinity column.

27. The method of claim 20, which further comprises passing the eluate from the affinity column over a desalting column.

28. The method of claim 20, which further comprises concentrating the protein.

29. The method of claim 28, wherein the protein is concentrated via filtration using pressure.

30. The method of claim 29, wherein the pressure is produced using a non-reactive gas.

31. The method of claim 30, wherein the non-reactive gas is argon or nitrogen.

32. The method of claim 28, wherein the protein is concentrated to a predetermined volume.

33. The method of claim 28, wherein the protein is concentrated to a predetermined concentration.

34. The method of claim 20, wherein the crude cell lysate is obtained by adding detergent to a cell culture and sonicating the cell culture, wherein the density of the cell culture upon harvesting is not significantly increased by external manipulation prior to sonication.

35. The method of claim 34, wherein the cell culture is not centrifuged prior to addition of the sonication.

36. The method of claim 20, wherein the affinity column contains resin functionalized with one or more of the following: Fe, Co, Ni, Cu, Zn, Al, amylose, Chitin, glutathione, protein A, protein G, an antibody, and an antibody fragment.

37. The method of claim 20, wherein the ion exchange resin is an anion exchange resin.

38. The method of claim 37, wherein the anion exchange resin is one or more of the following: diethylaminoethyl (DEAE), quaternary aminoethyl (QAE), and quaternary amine (Q) resin.

39. The method off claim 20, wherein the method is automated.

40. A system for preparing protein samples, comprising:
a filter for filtering a lysate;
at least one purifier fluidly coupled to the filter for purifying a protein within the filtered lysate; and
a concentrator fluidly coupled to the purifier for concentrating the purified protein, the concentrator using pressure to concentrate the purified protein.

41. The system of claim 40 further comprising:
a pump fluidly coupled to the filter, purifier and concentrator, the pump transferring the filtered lysate to the purifier and the purified protein to the concentrator.

42. The system of claim 40 further comprising:
at least one mixing module fluidly coupled to the purifier, the mixing module combining a composition with at least one of the filtered lysate and the purified protein.

43. The system of claim 40 further comprising:
a plurality of actuators selectively affecting the operation of at least one of the filter, purifier, and concentrator;
at least one processor executing instructions associated with the operation of at least one of the filter, purifier, and concentrator, the processor generating signals in response to the executed instructions; and
at least one controller communicating with the actuators in response to the signals generated by the processor.

44. The system of claim 43 further comprising:
a plurality of sensors sensing operational parameters of at least one of the filter, purifier, and concentrator; wherein the sensors communicate the operational parameters to the processor via the controller.

45. The system of claim 44 wherein at least one of the sensors detects at least one of a presence and a concentration of the purified protein being concentrated in the concentrator.

46. The system of claim 45 wherein the sensor is an ultraviolet sensor.

47. The system of claim 43 further comprising:
a control process operating within a memory of a digital data processing device, the control process providing the instructions executed by the processor.

48. The system of claim 47 wherein the control process controls the temperature and humidity of at least one of the filter, purifier, and concentrator.

49. The system of claim 48 wherein the temperature of the at least one of the filter, purifier, and concentrator is maintained between 2 and 4 degrees Celsius.

50. The system of claim 47 further comprising:
a messaging process communicatively coupled to the control process, the messaging process providing indicia of the operation of at least one of the filter, purifier, and concentrator to a remote user in response to a signal from the control process.

51. The system of claim 47 further comprising:
a display process communicatively coupled to the control process, the display process displaying indicia of the operation of at least one of the filter, purifier, and concentrator in a graphical user interface in response to a signal from the control process.

52. The system of claim 40 wherein the purifier comprises:
an ion exchange module binding charged molecules out of the filtered lysate to form an ion exchange flow through;
an affinity module binding a protein from the ion exchange flow through and eluting the bound protein using a buffer; and
a desalting module altering the buffer of the eluted protein to form the purified protein.

53. The system of claim 52 wherein the pressure used by the concentrator forces excess buffer from the purified protein, the amount of the excess buffer corresponding to at least one of a predetermined concentration of the purified protein and a predetermined volume of the excess buffer.

54. The system of claim 52 wherein the purifier comprises a second desalting module using gel filtration to fractionate macromolecules of the purified protein from impurities.

55. The system of claim 53 wherein the pressure is exerted by a compressed gas that is nonreactive with the purified protein.

56. The system of claim 40 wherein the concentrated protein exhibits a purity exceeding 95%.

57. The system of claim 40 further comprising:
at least one processor executing instructions associated with the operation of at least one of the filter, purifier, and concentrator, the processor resolving error conditions associated with the operation without an input from a user of the processor.

58. The system of claim 57 wherein the error condition corresponds to an obstruction in a tube associated with the fluid coupling of at least one of the filter, purifier, and concentrator.

59. The system of claim 40 further comprising:
at least one processor executing instructions associated with the operation of at least one of the filter, purifier, and concentrator, the processor affecting the operation to perform a self-cleaning process without an input from a user of the processor.

60. The system of claim 40 further comprising:
a plurality of channels for preparing a plurality of purified proteins in parallel, each of the channels including at least one filter, purifier, and concentrator for independently preparing a particular one of the plurality of proteins.

61. A system for purifying cell components, comprising:
a filter for filtering a lysate;
at least one purifier fluidly coupled to the filter for purifying a cell component within the filtered lysate; and
a concentrator fluidly coupled to the purifier for concentrating the purified cell component, the concentrator using pressure to concentrate the purified cell component.

62. A system for preparing protein samples, comprising:
a means for filtering a lysate;
a means for purifying a protein within the filtered lysate ; and
a means for concentrating the purified protein using pressure.

63. A method of preparing protein samples, comprising:
instructing a pump to transfer a filtered lysate to a protein purifier until a first sensor signal is received indicative of a level associated with the filtered lysate, the purifier purifying the filtered lysate into a protein solution;
instructing the pump to transfer the protein solution to a concentrator;
instructing a pressure valve to pressurize the concentrator until an amount of excess liquid is removed from the protein solution to form a concentrated solution; and
instructing the pressure valve to depressurize the concentrator in response to a signal from a second sensor.

64. The method of claim 63 further comprising:
instructing at least one of a plurality of flow valves to assume a particular configuration corresponding to the operation of the pump, the flow valve configuration affecting the operation of at least one of the purifier and the concentrator.

65. The method of claim 64 wherein the flow valve is controlled by a corresponding controller communicatively coupled to a software process, the software process specifying the flow valve configuration.

66. The method of claim 63 wherein the pump is controlled by a corresponding controller communicatively coupled to a software process, the software process specifying the operation of the pump.

67. The method of claim 63 further comprising:
transmitting a message identifying indicia of the operation of at least one of the purifier and concentrator to a remote user.

68. The method of claim 63 further comprising:
displaying indicia of the operation of at least one of the purifier and concentrator in a graphical user interface.

69. The method of claim 63 further comprising:
resolving an error condition associated with the operation of at least one of the purifier and concentrator without an input from a user.

70. The method of claim 63 further comprising:
cleaning at least one of the purifier and concentrator without an input from a user.

71. The method of claim 63 wherein purifying comprises:
binding charged molecules out of the filtered lysate to form a fractionated lysate;
binding a protein from the fractionated lysate and eluting the bound protein using a buffer; and
altering the buffer of the eluted protein to form the protein solution.

72. The method of claim 63 wherein the pressure valve uses a non-reactive gas to pressurize the concentrator.

73. The method of claim 63 wherein the second sensor detects at least one of a predetermined volume of the excess liquid and a predetermined concentration of the concentrated solution.

74. The method of claim 63 further comprising:
controlling the temperature and humidity of at least one of the purifier and the concentrator.

75. The method of claim 74 wherein the temperature of at least one of the purifier and the concentrator is maintained between 2 and 4 degrees Celsius.

76. The method of claim 63 further comprising:
providing a plurality of channels for preparing a plurality of concentrated solutions in parallel, each of the channels including at least one pump, purifier, and concentrator for independently preparing a particular one of the plurality of concentrated solutions.

77. A method of purifying cell components, comprising:
instructing a pump to transfer a filtered lysate to a purifier until a first sensor signal is received indicative of a level associated with the filtered lysate, the purifier purifying the filtered lysate into a cell component solution;
instructing the pump to transfer the cell component solution to a concentrator;
instructing a pressure valve to pressurize the concentrator until an amount of excess liquid is removed from the cell component solution to form a concentrated solution; and instructing the pressure valve to depressurize the concentrator in response to a signal from a second sensor.

78. A software application program for preparing protein samples, comprising:
a control process
instructing a pump to transfer a filtered lysate from a filter to a protein purifier until a first sensor signal is received indicative of a level associated with the filtered lysate, the purifier purifying the filtered lysate into a protein solution,
instructing the pump to transfer the protein solution to a concentrator,
instructing a pressure valve to pressurize the concentrator until an amount of excess liquid is removed from the protein solution to form a concentrated solution, and
instructing the pressure valve to depressurize the concentrator in response to a signal from a second sensor; and
a display process displaying indicia of the operation of at least one of the filter, purifier, and concentrator in a graphical user interface.

79. The program of claim 78 further comprising:
a messaging process providing indicia of the operation of at least one of the filter, purifier, and concentrator to a remote user in response to a signal from the control process.

80. The program of claim 78 wherein the control process instructs at least one of a plurality of flow valves to assume a particular configuration corresponding to the operation of the pump, the flow valve configuration affecting the operation of at least one of the filter, purifier, and concentrator.

81. The program of claim 78 wherein the control process resolves an error condition associated with the operation of at least one of the filter, purifier, and concentrator without an input from a user.

82. The program of claim 78 wherein the control process initiates a cleaning cycle of at least one of the filter, purifier, and concentrator without an input from a user.

83. The program of claim 78 wherein purifying comprises:
binding charged molecules out of the filtered lysate to form a fractionated lysate;
binding a protein from the fractionated lysate and eluting the bound protein using a buffer; and
altering the buffer of the eluted protein to form the protein solution.

84. The program of claim 78 wherein the pressure valve uses a non-reactive gas to pressurize the container.

85. The program of claim 78 wherein the second sensor detects at least one of a predetermined volume of the excess liquid and a predetermined concentration of the concentrated solution.

86. The program of claim 78 wherein the control process controls the temperature and humidity of at least one of the purifier and the concentrator.

87. The program of claim 78 wherein the control process instructs a plurality of channels to prepare a plurality of concentrated solutions in parallel, each of the channels including at least one pump, purifier, and concentrator for independently preparing a particular one of the plurality of concentrated solutions.

88. A system for preparing protein samples, comprising:
an array of filter columns;
an array of purifier columns in fluid communication with the array of filter columns;
an array of concentrators in fluid communication with the array of purifier columns; and
a plurality of channels for preparing a plurality of purified proteins in parallel, one or more channels including at least one of a filter column from the array of filter columns, at least one of a purifier column from the array of purifier columns, and at least one of a concentrator from the array of concentrators.

89. The system of claim 88 wherein at least some of the plurality of channels prepare purified proteins independently of each other.

90. The system of claim 88 wherein the array of purifier columns include:
an ion exchange column for binding charged molecules out of a lysate filtered by one of the filter columns to form an ion exchange flow through;
an affinity column binding a protein from the ion exchange flow through and eluting the bound protein using a buffer; and
a desalting column altering the buffer of the eluted protein to form the purified protein.

91. The system of claim 90, wherein one or more of the channels includes the ion exchange column, the affinity column, and the desalting column.

92. The system of claim 88 further comprising:
a plurality of actuators selectively affecting the operation of at least one of the filter columns, purifier columns, and concentrators;
at least one processor executing instructions associated with the operation of the at least one filter column, purifier column, and concentrator, the processor generating signals in response to the executed instructions; and
at least one controller communicating with the actuators in response to the signals generated by the processor.

93. The system of claim 92 further comprising:
a plurality of sensors sensing operational parameters of the at least one filter column, purifier column, and concentrator, wherein the sensors communicate the operational parameters to the processor via the controller.

94. The system of claim 93 wherein at least one of the sensors detects at least one of a presence and a concentration of the purified protein being concentrated in the concentrator.
